SEQUENCE LISTING (3 sequences)

SEQ ID NO: 1 - DNA, 36 bp, synthetic construct
  source 1..36: mol_type = other DNA
agctgcatgatgagctgcacggtggcagctcatcat

SEQ ID NO: 2 - DNA, 35 bp, synthetic construct
  source 1..35: mol_type = other DNA
ttggcccgcccaaaatctgtgattagatttgggcg

SEQ ID NO: 3 - DNA, 37 bp, synthetic construct
  source 1..37: mol_type = other DNA
tcaacatcagtctgataagctagtattatcagactga

(19) United States
(12) Patent Application Publication
(10) Pub. No.: US 2025/0369057 A1
(43) Pub. Date: Dec. 4, 2025

(54) METHOD FOR DETECTING HIGH SENSITIVITY OF CANCER CELL-DERIVED EXTRACELLULAR VESICLE GENE BY USING FUSION REACTION WITH LIPOSOMES

(71) Applicants: LABSPINNER, INC, Ulsan (KR); UNIST (Ulsan National Institute of Science and Technology, Ulsan (KR)

(72) Inventors: Kyusang LEE, Ulsan-si (KR); Beomseok LEE, Osan-si (KR); Jihye AHN, Ulsan (KR); Beomsun GO, Seoul (KR); Hanna KIM, Ulsan (KR); Yoon-Kyoung CHO, Ulsan-si (KR)

(21) Appl. No.: 19/235,274

(22) Filed: Jun. 11, 2025

Related U.S. Application Data
(63) Continuation-in-part of application No. PCT/KR2023/020308, filed on Dec. 11, 2023.

(30) Foreign Application Priority Data
Dec. 12, 2022 (KR) 10-2022-0172870

(51) Int. Cl. C12Q 1/6888; C12Q 1/6809

(52) U.S. Cl. CPC C12Q 1/6888; C12Q 1/6809; C12Q 2600/106; C12Q 2600/112; C12Q 2600/156; C12Q 2600/178

(57) ABSTRACT

The present invention successfully introduced a new approach to target specific EV subpopulations on the basis of charge-mediated fusion of EVs and CLIPs. By adjusting the surface charge of liposomes through the ratio of positively and negatively charged lipids, the optimal ratio that allows efficient and stable fusion with exosomes was confirmed. A method according to the present invention uses the advantages of a CLIP's high fusion rate, and rapid and broad applicability, and verified excellent sensitivity and selectivity for disease-derived EV miRNA in a lysis-free manner using droplet-microfluidics. Particularly, the EV-CLIP method enables digital detection of EGFR L858R and T790M mutations without pretreating a sample, and thus can simplify detection processes and prevent EV loss.

### CROSS-REFERENCE TO RELATED APPLICATIONS

This application is a continuation in part of PCT Application No. PCT/KR2023/020308, filed Dec. 11, 2023, which claims priority to and benefit of Korean Application No. 10-2022-0172870, filed Dec. 12, 2022, the entire contents of which are incorporated herein by reference.

### TECHNICAL FIELD

The present invention relates to a charged-liposome for detecting an extracellular vesicle (EV) and the use thereof, and more particularly, to a liposome for detecting an extracellular vesicle comprising a cationic lipid and a neutral lipid, and a composition for detecting a diseased cell-derived extracellular vesicle, a detection kit, a detection method, a composition for diagnosing diseases, a kit for diagnosing diseases, a method of providing information for diagnosing diseases, and a method of diagnosing diseases, comprising the liposome.

### SEQUENCE LISTING

The instant application contains a Sequence Listing which has been submitted electronically in XML file format and is hereby incorporated by reference in its entirety. Said XML copy, created on Oct. 25, 2023, is named PP-B2970 and is 4 gigabytes in size.

### BACKGROUND ART

The analysis of single extracellular vesicle (EV) has emerged as a powerful tool in the field of biomedical research, providing valuable insights into a variety of diseases and disorders (Sullivan, L. B., Nature Chemical Biology 2017, 13 (9), 924-925). This allows for detailed investigation of EV heterogeneity (Tkach, M.; Thery, C., Cell 2016, 164 (6), 1226-1232), identification of disease-specific biomarkers (Peinado, H., et al., Nature Medicine 2012, 18 (6), 883-891), and monitoring of dynamic changes in disease progression by identifying unique nucleic acids (Cocks, A., et al., Seminars in Cancer Biology 2021, 75, 127-135), proteins (Whittle, K., et al., Critical Reviews in Oncology/Hematology 2022, 171, 103603), or other molecules (Yu, W., et al., Ann Oncol 2021, 32 (4), 466-477), which are indicative of a particular disease. Therefore, investigating the contents and characteristics of single EV has the potential to reveal important information about disease processes (Marar, C., et al., Nature Immunology 2021, 22 (5), 560-570). In order to detect EV-derived RNA with high sensitivity, the optimal standard quantitative reverse transcription-polymerase chain reaction (qRT-PCR) (Gandham, S., et al., Trends Biotechnol 2020, 38 (10), 1066-1098) and various novel approaches have been proposed (Shao, H., et al., Chem Rev 2018, 118 (4), 1917-1950). Despite the notable advantages such methods offer, most of these detection methods still present challenges that require attention, especially due to the inclusion of laborious and time-consuming steps such as EV isolation from biological samples such as plasma, EV lysis, RNA extraction, and reverse transcription amplification (Erdbrugger, U.; Lannigan, J., Cytometry Part A 2016, 89 (2), 123-134). In addition, since biological samples contain a mixture of EVs derived from tumor cells and EVs derived from non-tumor cells and also since conventional methods for RNA isolation and analysis from bulk solutions are performed, distinguishing tumor cell-derived RNA signals continues to be a challenging task (Bordanaba-Florit, G., et al., Nature Protocols 2021, 16 (7), 3163-3185). Consequently, further research is essential to develop more efficient and accurate strategies for detection of tumor cell-derived EV RNA.

Membrane fusion mediated by SNARE proteins and often facilitated by calcium ions (Ca^{2±}), is essential for diverse cellular processes including exocytosis and endocytosis, membrane remodeling, cell division, signal transduction, and intracellular transport (Koike, S.; Jahn, R., Nature Communications 2019, 10 (1), 1608). Extensive research has been conducted on fusion mechanisms of phospholipid compartments (Ma, M.; Bong, D., Accounts of Chemical Research 2013, 46 (12), 2988-2997) and has been applied to develop functional systems that may be applied in diagnosis and therapy (Mazur, F.; Chandrawati, R., ChemNanoMat 2021, 7 (3), 223-237). There have been devised various EV membrane fusion processes such as pH-dependent (Yang, Y., et al., Advanced Materials 2017, 29 (13), 1605604), polyethylene glycol-mediated (Piffoux, M., et al., ACS Nano 2018, 12 (7), 6830-6842), catechol-metal supramolecular complex (Kumar, S., et al., Nature Catalysis 2021, 4 (9), 763-774), freeze-thaw cycle-mediated (Cheng, L., et al., Biomaterials 2021, 275, 120964), and DNA zipper-mediated (Peruzzi, J. A., et al., Angew Chem Int Ed Engl 2019, 58 (51), 18683-18690) processes. Such processes involve a variety of molecular compositions on the plasma membrane that bind or dock to the membrane, bringing them into close proximity while inducing local disturbances, thus reducing the energy barrier for fusion. Recently, many strategies have been developed to utilize fusogenic vesicles inspired by viral infection mechanisms (Gao, X., et al., Angewandte Chemie International Edition 2019, 58 (26), 8719-8723) and aptamer-mediated fusion (Feng, J., et al., Analytical Chemistry 2023, 95 (19), 7743-7752) for detection of EV RNAs within natural environments. However, these methods also require complex genetic manipulations or optimization of numerous aptamers, which may be technically challenging and time-consuming, limiting utility thereof in a broad range of clinical scenarios. Therefore, it is important to develop a more generalized and simplified approach for tumor-derived EV RNA detection that can be easily applied in clinical practice.

In the present invention, the inventors successfully developed a simple, efficient, surface protein-independent, charge-induced fusion method for EV/liposome fusion within a microfluidic droplet reactor, enabling for the investigation of miRNAs and mRNAs inside individual EVs. By manipulation of the ratio of positively and negatively charged lipids, the surface charge of liposomes could be finely tuned to enable efficient fusion with EVs, with precise control of fusion ranging from less than 5% to over 60%. A certain combination of charged liposomes showed the highest fusion efficiency among different charge types. Furthermore, the present invention enables high-throughput single-vesicle miRNA or mRNA profiling by sorting individual EVs in emulsion droplets and utilizing charged-liposome (CLIP) EV detection (EV-CLIP) via droplet scanning. The present inventors successfully digitally detected EGFR L858R and T790M mutations from blood plasma samples from 73 lung cancer patients (17 without mutation, 56 with mutation) and 10 healthy donors. In particular, the innovative EV-CLIP method minimized EV loss by simplifying the detection process without sample preprocessing.

The above information in this Background Art is intended only to improve the understanding of the background of the present invention and it may not include information that constitutes prior art known to one having ordinary skill in the art to which the present invention pertains.

### SUMMARY OF THE INVENTION

It is an object of the present invention to provide a charged-liposome for detecting a cancer cell-derived extracellular vesicle (EV) gene with high sensitivity and high selectivity.

It is another object of the present invention to provide a composition for detecting a cancer cell-derived extracellular vesicle comprising the liposome, a detection kit, a detection method, a composition for diagnosing cancer, a kit for diagnosing cancer, a method of providing information for diagnosing cancer, and a method of diagnosing cancer.

In order to accomplish the above objects, the present invention provides a liposome for detecting a cancer cell-derived extracellular vesicle (EV) comprising a cationic lipid and a neutral lipid, in which a cancer cell-specific molecular beacon is encapsulated within the liposome.

The present invention also provides a composition and kit for detecting a cancer cell-derived extracellular vesicle comprising the liposome for detecting an extracellular vesicle.

The present invention also provides a method of detecting a cancer cell-derived extracellular vesicle comprising fusing the liposome for detecting an extracellular vesicle with an extracellular vesicle derived from a biological sample.

The present invention also provides a composition and kit for diagnosing cancer comprising the liposome for detecting an extracellular vesicle.

The present invention also provides a method of providing information for cancer diagnosis and a method of diagnosing cancer comprising fusing the liposome for detecting an extracellular vesicle with an extracellular vesicle derived from a biological sample.

In one aspect, the present disclosure provides a liposome for detecting a pathological cell-derived extracellular vesicle (EV) from a subject with a neurodegenerative disease comprising a cationic lipid and a neutral lipid, in which a pathological cell-specific molecular beacon is encapsulated within the liposome. In some embodiments, the cationic lipid is selected from the group consisting of 1,2-dioleoyl-3-trimethylammonium propane (DOTAP), 1,2-dioleyloxy-3-dimethylamino-propane (DODMA), 1,2-di-O-octadecenyl-3-trimethylammonium propane (DOTMA), 3β-[N-(N′,N′-dimethylaminoethane)-carbamoyl]cholesterol (DC-Chol), dimethyldioctadecylammonium bromide (DODAB), 1,2-dioleoyl-3-dimethylammonium-propane (DODAP), dioctadecyldimethyl ammonium chloride (DODAC), 1,2-distearyloxy-N,N-dimethyl-3-aminopropane (DSDMA), 1,2-dimyristyloxy-propyl-3-dimethyl-hydroxyethyl ammonium bromide (DMRIE), 1,2-dimyristoyl-sn-glycero-3-ethylphosphocholine (DMEPC), 1,2-dimyristoyl-3-trimethylammonium propane (DMTAP), 1,2-diolcoyl-3-dimethyl-hydroxyethyl ammonium bromide (DORIE), 2,3-dioleyloxy-N-[2-(sperminecarboxamido) ethyl]-N,N-dimethyl-1-propanaminium (DOSPA), 1,2-dilinoleyloxy-N,N-dimethyl-3-aminopropane (DLinDMA), 1,2-dilinolenyloxy-N,N-dimethyl-3-aminopropane (DLenDMA), dioctadecylamidoglycylspermine (DOGS), 3-dimethylamino-2-(cholest-5-en-3-β-oxybutan-4-oxy)-1-(cis,cis-9,12-octadecadienoxy) propane (CLinDMA), 2-[5′-(cholest-5-en-3-β-oxy)-3′-oxapentoxy]-3-dimethyl-1-(cis,cis-9′, 12′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-dilinoleoyloxy-N,N-dimethylpropylamine (DLinDAP), 1,2-N,N′-dilinoleylcarbamyl-3-dimethylaminopropane (DLincarbDAP), 1,2-dilinoleoylcarbamyl-3-dimethylaminopropane (DLinCDAP), 2,2-dilinoleyl-4-dimethylaminomethyl-[1,3]-dioxolane (DLin-KDMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino) butanoate (DLin-MC3-DMA), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), (±)-N-(2-aminoethyl)-N,N-dimethyl-2,3-bis(tetradecyloxy)-1-propanaminium bromide (BAE-DMRIE), N-(4-carboxybenzyl)-N,N-dimethyl-2,3-bis(oleoyloxy) propan-1-aminium (DOBAQ), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido) ethyl]-3,4-di[oleyloxy]-benzamide (MVL5), 1,2-diolcoyl-sn-glycero-3-ethylphosphocholine (DOEPC), 2,3-bis(dodecyloxy)-N-(2-hydroxyethyl)-N,N-dimethylpropan-1-aminium bromide (DLRIE), N-(2-aminoethyl)-N,N-dimethyl-2,3-bis(tetradecyloxy) propan-1-aminium bromide (DMORIE), di((Z)-non-2-en-1-yl) 8,8′-((((2 (dimethylamino)ethyl) thio) carbonyl) azanediyl) dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), di((Z)-non-2-en-1-yl)-9-((4-(dimethylaminobutanoyl)oxy) heptadecanedioate (L319), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino) propionamide (lipidoid 98N12-5), and 1-[2-[bis(2-hydroxydodecyl)amino]ethyl-[2-[4-[2-[bis(2-hydroxydodecyl)amino]ethyl]piperazin-1-yl]ethyl]amino]dodecan-2-ol (lipidoid C12-200). In some embodiments, the neutral lipid is selected from the group consisting of 1,2-dioleoyl-sn-glycero-3-phosphocholine (DOPC), 1,2-dimyristoyl-sn-glycero-3-phosphocholine (DMPC), 1,2-dioleoyl-sn-glycero-3-phosphoethanolamine (DOPE), 1,2-dipalmitoyl-sn-glycero-3-phosphocholine (DPPC), 1,2-distearoyl-sn-glycero-3-phosphocholine (DSPC), 1,2-dilinoleoyl-sn-glycero-3-phosphocholine (DLPC), phosphatidylserine (PS), phosphatidylethanolamine (PE), phosphatidylglycerol (PG), phosphoric acid (PA), and phosphatidylcholine (PC). In some embodiments, the cationic lipid is 1,2-dioleoyl-3-trimethylammonium propane (DOTAP) and the neutral lipid is 1,2-dioleoyl-sn-glycero-3-phosphocholine (DOPC). In some embodiments, a molar ratio (%) of the cationic lipid in the liposome is 25 to 75%. In some embodiments, the neurodegenerative disease is selected from the group consisting of Huntington's disease, Alzheimer's disease, and Parkinson's disease. In some embodiments, the molecular beacon comprises a nucleic acid and a fluorophore. In some embodiments, the nucleic acid comprises a sequence encoding one or more of SEQ ID NOs: 4-9.

In another aspect, the present disclosure provides a composition for detecting a pathological cell-derived extracellular vesicle comprising the liposome according to any of the preceding embodiments.

In a different aspect, the present disclosure provides a kit for detecting a pathological cell-derived extracellular vesicle comprising the liposome according to any of the preceding embodiments.

In another aspect, the present disclosure provides a method of detecting a pathological cell-derived extracellular vesicle, comprising: fusing the liposome of the preceding embodiments with an extracellular vesicle derived from a biological sample; and determining that the extracellular vesicle is a pathological cell-derived extracellular vesicle when a fluorescence signal is generated. In some embodiments, the fusing is performed within a droplet reactor comprising two aqueous phase channels, one oil phase channel, one junction, and one outlet channel. In some embodiments, aqueous droplets are generated at the junction by introducing the liposome and the extracellular vesicle derived from the biological sample respectively into the two aqueous phase channels.

In one aspect, the present disclosure provides a diagnostic composition comprising the liposome according to any of the preceding embodiments.

In a different aspect, the present disclosure provides a kit comprising the liposome according to any of the preceding embodiments and instructions for use thereof. In some embodiments, the instructions for use comprise diagnosing a neurodegenerative disease selected from the group consisting of Huntington's disease, Alzheimer's disease, and Parkinson's disease.

In another aspect, the present disclosure provides a method of selecting a subject having neurodegenerative disease for treatment, comprising: fusing the liposome according to any of the preceding embodiments with an extracellular vesicle derived from a biological sample; and selecting the subject for treatment when a fluorescence signal is generated. In some embodiments, the fusing is performed within a droplet reactor comprising two aqueous phase channels, one oil phase channel, one junction, and one outlet channel. In some embodiments, the neurodegenerative disease is selected from the group consisting of Huntington's disease, Alzheimer's disease, and Parkinson's disease.

In one aspect, the present disclosure provides a liposome for detecting a cancer cell-derived extracellular vesicle (EV) comprising a cationic lipid and a neutral lipid, in which a cancer cell-specific molecular beacon is encapsulated within the liposome. In some embodiments, the cationic lipid is selected from the group consisting of 1,2-dioleoyl-3-trimethylammonium propane (DOTAP), 1,2-dioleyloxy-3-dimethylamino-propane (DODMA), 1,2-di-O-octadecenyl-3-trimethylammonium propane (DOTMA), 3B-[N-(N′,N′-dimethylaminoethane)-carbamoyl]cholesterol (DC-Chol), dimethyldioctadecylammonium bromide (DODAB), 1,2-dioleoyl-3-dimethylammonium-propane (DODAP), dioctadecyldimethyl ammonium chloride (DODAC), 1,2-distearyloxy-N,N-dimethyl-3-aminopropane (DSDMA), 1,2-dimyristyloxy-propyl-3-dimethyl-hydroxyethyl ammonium bromide (DMRIE), 1,2-dimyristoyl-sn-glycero-3-ethylphosphocholine (DMEPC), 1,2-dimyristoyl-3-trimethylammonium propane (DMTAP), 1,2-dioleoyl-3-dimethyl-hydroxyethyl ammonium bromide (DORIE), 2,3-dioleyloxy-N-[2-(sperminecarboxamido) ethyl]-N,N-dimethyl-1-propanaminium (DOSPA), 1,2-dilinoleyloxy-N,N-dimethyl-3-aminopropane (DLinDMA), 1,2-dilinolenyloxy-N,N-dimethyl-3-aminopropane (DLenDMA), dioctadecylamidoglycylspermine (DOGS), 3-dimethylamino-2-(cholest-5-en-3-β-oxybutan-4-oxy)-1-(cis,cis-9,12-octadecadienoxy) propane (CLinDMA), 2-[5′-(cholest-5-en-3-β-oxy)-3′-oxapentoxy]-3-dimethyl-1-(cis,cis-9′,12′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-dilinoleoyloxy-N,N-dimethylpropylamine (DLinDAP), 1,2-N,N′-dilinoleylcarbamyl-3-dimethylaminopropane (DLincarbDAP), 1,2-dilinoleoylcarbamyl-3-dimethylaminopropane (DLinCDAP), 2,2-dilinoleyl-4-dimethylaminomethyl-[1,3]-dioxolane (DLin-KDMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino) butanoate (DLin-MC3-DMA), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), (±)-N-(2-aminoethyl)-N,N-dimethyl-2,3-bis(tetradecyloxy)-1-propanaminium bromide (BAE-DMRIE), N-(4-carboxybenzyl)-N,N-dimethyl-2,3-bis(oleoyloxy) propan-1-aminium (DOBAQ), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido) ethyl]-3,4-di[olcyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-aminium bromide (DLRIE), N-(2-aminoethyl)-N,N-dimethyl-2,3-bis(tetradecyloxy) propan-1-aminium bromide (DMORIE), di((Z)-non-2-en-1-yl) 8,8′-((((2 (dimethylamino)ethyl) thio) carbonyl) azanediyl) dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), di((Z)-non-2-en-1-yl)-9-((4-(dimethylaminobutanoyl)oxy) heptadecanedioate (L319), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino) propionamide (lipidoid 98N12-5), and 1-[2-[bis(2-hydroxydodecyl)amino]ethyl-[2-[4-[2-[bis(2-hydroxydodecyl)amino]ethyl]piperazin-1-yl]ethyl]amino]dodecan-2-ol (lipidoid C12-200). In some embodiments, the neutral lipid is selected from the group consisting of 1,2-diolcoyl-sn-glycero-3-phosphocholine (DOPC), 1,2-dimyristoyl-sn-glycero-3-phosphocholine (DMPC), 1,2-dioleoyl-sn-glycero-3-phosphoethanolamine (DOPE), 1,2-dipalmitoyl-sn-glycero-3-phosphocholine (DPPC), 1,2-distearoyl-sn-glycero-3-phosphocholine (DSPC), 1,2-dilinoleoyl-sn-glycero-3-phosphocholine (DLPC), phosphatidylserine (PS), phosphatidylethanolamine (PE), phosphatidylglycerol (PG), phosphoric acid (PA), and phosphatidylcholine (PC). In some embodiments, the cationic lipid is 1,2-dioleoyl-3-trimethylammonium propane (DOTAP) and the neutral lipid is 1,2-dioleoyl-sn-glycero-3-phosphocholine (DOPC). In some embodiments, a molar ratio (%) of the cationic lipid in the liposome is 25 to 75%.

In a different aspect, the present disclosure provides a composition for detecting a cancer cell-derived extracellular vesicle comprising the liposome according to the preceding embodiments.

In one aspect, the present disclosure provides a kit for detecting a cancer cell-derived extracellular vesicle comprising the liposome according to the preceding embodiments.

In another aspect, the present disclosure provides a method of detecting a cancer cell-derived extracellular vesicle, comprising: fusing the liposome according to the preceding embodiments with an extracellular vesicle derived from a biological sample; and determining that the extracellular vesicle is a cancer cell-derived extracellular vesicle when a fluorescence signal is generated. In some embodiments, the fusing is performed within a droplet reactor comprising two aqueous phase channels, one oil phase channel, one junction, and one outlet channel. In some embodiments, aqueous droplets are generated at the junction by introducing the liposome and the extracellular vesicle derived from the biological sample respectively into the two aqueous phase channels.

In a different aspect, the present disclosure provides a composition for diagnosing cancer comprising the liposome according to the preceding embodiments. In some embodiments, the cancer is selected from the group consisting of squamous cell carcinoma, small cell lung cancer, non-small cell lung cancer, lung cancer, peritoneal cancer, colon cancer, bile duct tumor, nasopharyngeal cancer, laryngeal cancer, bronchial cancer, thyroid cancer, oral cancer, osteosarcoma, gallbladder cancer, bile duct cancer, kidney cancer, bladder cancer, renal cell cancer, melanoma, brain cancer, glioma, glioblastoma, brain tumor, skin cancer, pancreatic cancer, breast cancer, liver cancer, bone marrow cancer, small intestine cancer, esophageal cancer, large intestine cancer, stomach cancer, eye cancer, urethral cancer, cervical cancer, prostate cancer, ovarian cancer, metastatic cancer, head and neck cancer, rectal cancer, non-Hodgkin's lymphoma, multiple myeloma, acute myelogenous leukemia, lymphoma, acute lymphoblastic leukemia, and chronic myelogenous leukemia.

In another aspect, the present disclosure provides a kit for diagnosing cancer comprising the liposome according to the preceding embodiments. In some embodiments, the cancer is selected from the group consisting of squamous cell carcinoma, small cell lung cancer, non-small cell lung cancer, lung cancer, peritoneal cancer, colon cancer, bile duct tumor, nasopharyngeal cancer, laryngeal cancer, bronchial cancer, thyroid cancer, oral cancer, osteosarcoma, gallbladder cancer, bile duct cancer, kidney cancer, bladder cancer, renal cell cancer, melanoma, brain cancer, glioma, glioblastoma, brain tumor, skin cancer, pancreatic cancer, breast cancer, liver cancer, bone marrow cancer, small intestine cancer, esophageal cancer, large intestine cancer, stomach cancer, eye cancer, urethral cancer, cervical cancer, prostate cancer, ovarian cancer, metastatic cancer, head and neck cancer, rectal cancer, non-Hodgkin's lymphoma, multiple myeloma, acute myelogenous leukemia, lymphoma, acute lymphoblastic leukemia, and chronic myelogenous leukemia.

In a one aspect, the present disclosure provides a method of providing information for diagnosing cancer, comprising: fusing the liposome of the preceding embodiments with an extracellular vesicle derived from a biological sample; and determining that the sample is cancerous when a fluorescence signal is generated. In some embodiments, the fusing is performed within a droplet reactor comprising two aqueous phase channels, one oil phase channel, one junction, and one outlet channel. In some embodiments, the cancer is selected from the group consisting of squamous cell carcinoma, small cell lung cancer, non-small cell lung cancer, lung cancer, peritoneal cancer, colon cancer, bile duct tumor, nasopharyngeal cancer, laryngeal cancer, bronchial cancer, thyroid cancer, oral cancer, osteosarcoma, gallbladder cancer, bile duct cancer, kidney cancer, bladder cancer, renal cell cancer, melanoma, brain cancer, glioma, glioblastoma, brain tumor, skin cancer, pancreatic cancer, breast cancer, liver cancer, bone marrow cancer, small intestine cancer, esophageal cancer, large intestine cancer, stomach cancer, eye cancer, urethral cancer, cervical cancer, prostate cancer, ovarian cancer, metastatic cancer, head and neck cancer, rectal cancer, non-Hodgkin's lymphoma, multiple myeloma, acute myelogenous leukemia, lymphoma, acute lymphoblastic leukemia, and chronic myelogenous leukemia.

### BRIEF DESCRIPTION OF THE DRAWINGS

FIG. 1 shows an overview of EV RNA analysis based on EV-CLIP fusion reaction. (a) Schematic of a process for inducing a fusion reaction between CLIP loaded with a molecular beacon (MB) constructed for the RNA to be analyzed and EV in a droplet reactor and detecting specific gene mutations within EV. For example, MBs for detecting gene EGFR L858R mutation, which is associated with targeted therapy selection for lung cancer patients, are tagged with a green fluorescent molecule, and MBs for EGFR T790M, which is associated with drug resistance, are tagged with a red fluorescent molecule, and thus, when the mutant genes and MBs react within the EVs in each droplet reactor, a fluorescence signal is generated, and the number of droplets in which the fluorescence signal is detected is counted and used for digital quantitative analysis of EV-derived RNA. (b) Schematic of the CLIP (charged-liposome) preparation using microfluidic hydrodynamic focusing method. The CLIP is composed of positively charged 1,2-dioleoyl-3-trimethylammonium propane (DOTAP, red) and negatively charged 1,2-dioleoyl-sn-glycero-3-phosphocholine (DOPC, green) lipids. (c) Representative image of the CLIP taken using transmission electron microscope (TEM) (diameter: 92.5±5.65 nm, n=3 with 9 data points). Scale bar: 100 nm. (d) Zeta potential of CLIPs with respect to various molar ratio compositions (%) of DOTAP and DOPC (^{χ}DOTAP: ^{χ}DOPC) (0% DOTAP: −18.63±0.76, 25% DOTAP: 16.17±3.05, 50% DOTAP: 26.90±5.27, 75% DOTAP: 32.20±2.26, 100% DOTAP: 38.67±2.51), in which data are presented as mean±SD and are based on three independently synthesized CLIP batches. (e) Hydrodynamic size (D_{H}) distribution of CLIPs with different ratio of DOTAP, in which size and surface charge measurements were performed using DLS (0% DOTAP: 95.82±8.87 nm, 25% DOTAP: 116.40±3.67 nm, 50% DOTAP: 113.40±1.00 nm, 75% DOTAP: 115.03±8.86 nm, 100% DOTAP: 113.7±4.3 nm). Data are presented as mean±SD and are based on three independently synthesized CLIP batches. All statistical analyses were performed by one-way ANOVA.

FIG. 2 shows an experimental setup for Microfluidic Hydrodynamic Focusing (MHF)-based liposome generation. The setup includes a digital microscope, a chip stand, oil and aqueous phase pumps, a computer, and a 5-input chip. Lipids dissolved in ethanol (EtOH) are loaded into the oil phase pump, PBS is loaded into the aqueous phase pump. The oil pump propels the lipids to the left side of the chip, while the aqueous pump propels the PBS to the right side of the chip. With proper flow control, the oil layer is interposed between two aqueous layers, resulting in the formation of liposomes within the 5-input chip.

FIG. 3 shows size distribution of liposomes measured by nanoparticle tracking analysis (NTA). The liposomes showed a single peak distribution curve, indicating a homogeneous liposome population.

FIG. 4 shows stability of various CLIPs over time. In order to evaluate the size change of liposomes over 48 hours, the size was measured over time. The size of 0%, 25%, 50%, and 75% DOTAP remained stable over time, whereas the size of 100% DOTAP continuously increased up to 300 nm after 48 hours (6 hr: 160.53±8.15 nm, 12 hr: 188.27±7.58 nm, 24 hr: 230.37±14.96 nm, 48 hr: 311.50±8.51 nm).

FIG. 5 shows EV isolation. (a) ExoDisc platform, a centrifugal disc equipped with anodized aluminum oxide filters with a pore diameter of 0.02 μm (Exodisc, LabSpinner). The supernatant was passed through the filter by centrifugation at 3,000 rpm (approximately 500 g), and 100 μl of concentrated EVs from the collection chamber was resuspended in 1×PBS with a dilution factor of 2. (b) Tabletop centrifuge platform for ExoDisc operation. (c) A principle of ExoDisc operation. (d) The EVs were characterized by nanoparticle tracking analysis (NTA).

FIG. 6 shows an experimental setup for droplet generation. The setup consists of a digital microscope, a chip stand, an oil pump, two sample pumps, a sample reservoir, a droplet outlet, a droplet chip, a computer, and a droplet reservoir chip. EVs and CLIPs are loaded separately into the two sample pumps, and the FC-40 surfactant is loaded into the oil pump and the sample reservoir. The oil phase pump pushes the FC-40 into the vertical side of the T-junction, and the sample pumps push EVs and CLIPs into the sample channel, and the EVs and CLIPs meet in the T-junction to form water-in-oil droplets, which are then collected at the droplet outlet. Then, the droplets are imaged for analysis by using the droplet reservoir chip under a fluorescence microscope.

FIG. 7 shows controlled fusion of EVs and CLIPs. (a) Schematic of CLIP and EV fusion using a droplet generation chip. The microfluidic device was designed to generate water-in-oil droplet reactors at the flow-focusing junction, where two aqueous phases (EVs and CLIPs) met and were transferred into an oil stream (surfactant PFPE-PEG in FC-40) to form droplets. (b) Schematic of Forster resonance energy transfer (FRET)-based lipid mixing assay to analyze the fusion reaction of EVs and CLIPs. The average distance between FRET-pair donor (green, nitrobenzoxadiazole) and acceptor (red, rhodamine) lipid probes increases following charge-based fusion of a labeled CLIP membrane and unlabeled EV membrane, resulting in decreased FRET efficiency (PE: phosphoethanolamine; PS: phosphatidylserine). (c) The results of analysis of the extent of lipid mixing in fused vesicles with respect to the fusion ratio of EVs and CLIPs and the percentage of DOTAP in the CLIP composition. All statistical analyses were performed by one-way ANOVA using Dunnett's multiple comparison test. (d) Confocal laser scanning microscope (CLSM) images of EVs, CLIPs, and fused vesicles, indicating colocalization of EVs (green) and CLIPs (red). With high-resolution confocal laser scanning microscope (CLSM) images of EVs, CLIPs, and fused vesicles (EV-CLIP) in three different ratios of EVs to CLIPs (75% DOTAP) (molar ratios 10:1, 1:1, and 1:10). Intensity analysis shows colocalization of EVs (green) and CLIPs (red). (c) A graph showing the ratio of each vesicle in the analysis results of (d). Analysis was conducted from 10 independent images, with vesicle counts ranging from 100 to 160. Statistical analysis was performed by one-way ANOVA. (f) Representative images of a CLIP, an EV, and a fused vesicle taken using the transmission electron microscope (TEM) (diameter of fused vesicle: 174.85±13.83 nm, n=5). scale bar: 100 nm. (g) Size of vesicles before and after controlled fusion (EVs: 137.00±14.30 nm, CLIPs: 115.03±8.86 nm, EV-CLIPs: 174.10±10.28), in which data represent mean±SD, n=3. All statistical analyses were performed by one-way ANOVA using Tukey's multiple comparison test. (h) Zeta potential of vesicles before and after controlled fusion, in which data represent mean±SD, n=3. All statistical analyses were performed by one-way ANOVA using Tukey's multiple comparison test.

FIG. 8 shows droplets size distribution graph, in which droplets were measured using the Droplet Monitor application. It is confirmed that the average size of droplets was determined to be 27.40±2.06 μm.

FIG. 9 shows an increase in size and zeta potential of the fused vesicles with respect to the percentage of DOTAP. (a) A graph showing an increase in size of the fused vesicles with respect to the percentage of DOTAP (0% DOTAP: 135.9±24.7 nm, 25% DOTAP: 150.5±24.3 nm, 50% DOTAP: 161.6±11.9 nm, 75% DOTAP: 174.1±10.3 nm, 100% DOTAP: 231.3±42.8 nm). Each data point represents mean±SD, n=3. All statistical analyses were performed by one-way ANOVA. (b) A graph showing a change in surface charge of the fused vesicles with respect to the percentage of DOTAP (0% DOTAP: −18.4±6.9, 25% DOTAP: −12.3±3.0, 50% DOTAP: −9.24±4.55, 75% DOTAP: −3.74±3.80, 100% DOTAP: −0.71±4.39). Each data point represents mean±SD, n=3. All statistical analyses were performed by one-way ANOVA.

FIG. 10 shows Pearson correlation coefficient (PCC) values of the fused vesicles, liposomes, and EVs. Pearson correlation coefficient (PCC) was measured in 10 different single vesicles and calculated using the JaCoP algorithm in ImageJ. It is confirmed that PCC of the fused vesicles was 0.690±0.096, PCC of liposomes was-0.0139±0.01264, and PCC of EVs was 0.0021±0.0066.

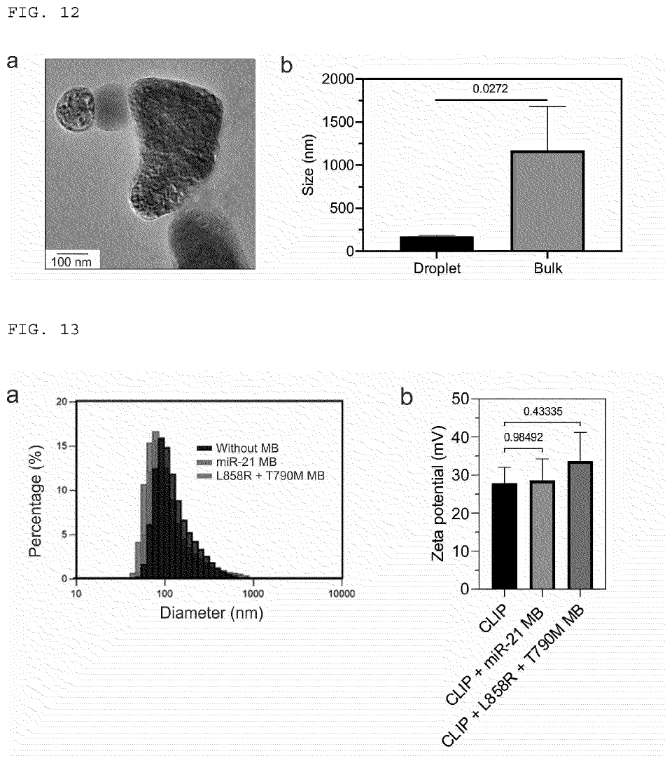

FIG. 11 shows effect of Rho-NBD addition to liposomes in terms of zeta potential. It shows zeta potential changes of liposomes with different ratio of DOTAP (0%, 25%, 75%, and 100% DOTAP). Each data point represents mean±SD, n=3. All statistical analyses were performed using two-tailed unpaired Student's t-test.

FIG. 12 shows TEM image and size comparison of aggregates formed during EV-liposome fusion in bulk-scale. (a) TEM image showing aggregates formed when EV-liposome fusion occurs in bulk scale. Scale bar: 100 nm. (b) Comparison of the sizes of fused vesicles in droplet and bulk scale. The average size of fused vesicles in droplets is 174.1±10.28 nm, the average size of fused vesicles in bulk scale is 1173.17±508.58 nm. Each data point represents mean±SD, n=3. All statistical analyses were performed using two-tailed unpaired Student's t-test.

FIG. 13 shows effect of MB insertion into the liposomes in terms of size and zeta potential. (a) A graph showing size distribution of three different liposome populations (without molecular beacon (MB); with miR-21-detecting MB; and with EGFR mutation-detecting MB). (b) A graph showing zeta potential of three different liposome populations (without molecular beacon (MB); with miR-21 detecting MB; and with EGFR mutation detecting MB). Each data point represents mean±SD, n=3. All statistical analyses were performed by one-way ANOVA.

FIG. 14 shows molecular beacon-based tumor EV detection. (a) Schematic of CLIP generation using an MHF chip, fusion reaction with EVs using a droplet reactor, and detection using a detection chamber chip. (b) A photograph showing the three chips. (c) Representative fluorescence images of miR-21 molecular beacon-based detection of various concentrations of H1975 cells-derived EVs in phosphate-buffered saline (PBS). (d) Results of miR-21 detection with respect to the concentration of H1975 cells-derived EVs, data representing mean±SD, n=3, (e) the limit of detection (LOD) was found to be 79 EVs/μl. (f) A graph showing results of miR-21 molecular beacon-based detection in normal persons and cancer patients, confirming differences therebetween.

FIG. 15 shows molecular beacon-based tumor EV detection. (a) Representative fluorescence images of L858R, T70M, and L858R±T790M molecular beacon-based detection of H1975 cells-derived EVs in phosphate-buffered saline (PBS). (b) Results of detection of L858R and T790M mutations with respect to the concentration of H1975 cells-derived EVs in phosphate-buffered saline (PBS). Data represent mean±SD, n=3, and limit of detection (LOD) was 1348 EVs/μl and 3595 EVs/μl, respectively. (c) Results of detection of L858R and T790M mutations with respect to the concentration of H1975 cells-derived EVs in human plasma. Data represent mean±SD, n=3, and limit of detection (LOD) was 1348 EVs/μl and 2926 EVs/μl, respectively. (d) Results of Droplet Digital Polymerase Chain Reaction (ddPCR) performed after EV isolation. Each data point represents mean±SD, n=3. (c) and (f) Correlation of the detection readouts of EGFR L858R mutation and T790M mutation in PBS and human plasma, in which data represent mean±SD from independent analysis, n=3.

FIG. 16 shows a flowchart of droplet image processing. The flowchart describes the processing steps of droplet image for the analysis, starting from the image acquisition, to processing, quantification and analysis

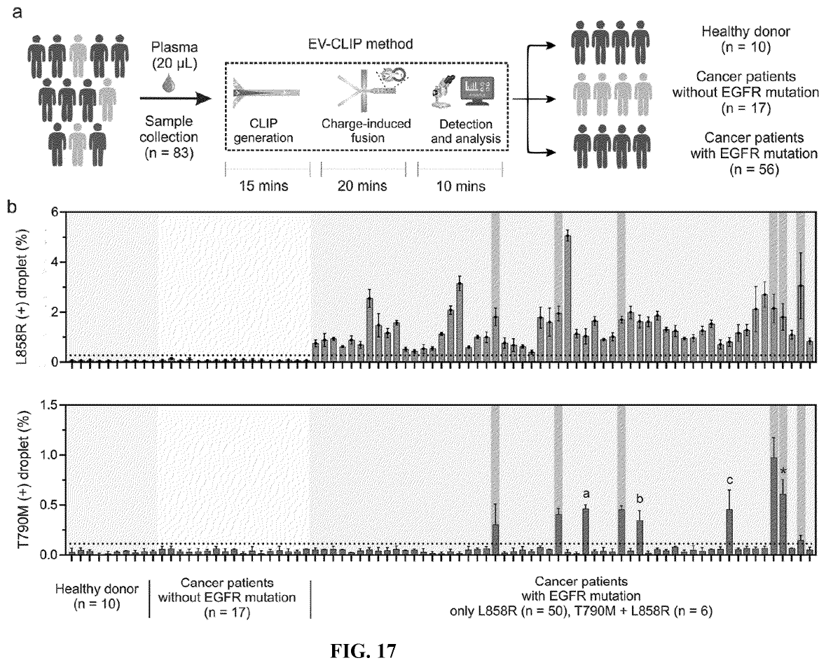

FIG. 17 shows detection of EGFR L858R and T790M mutations from lung cancer patient samples using a fusion system. (a) Schematic of the detection of EGFR L858R and T790M mutations from 10 healthy donors and 73 lung cancer patients using EV-CLIP fusion from plasma samples of normal persons and patients. (b) L858R mutation and T790M mutation droplet detection % from 10 healthy donors, 17 lung cancer patients without EGFR mutation, and 56 lung cancer patients with EGFR mutation. (c) Comparison of detection of L858R and T790M mutations by group. (d) ROC (Receiver Operating Characteristic) curves for detection of L858R and T790M mutations, in which AUC (Area Under the Curve) was shown to be 1.0 for L858R and 0.9762 for T790M. Each data point represents the average of three independent repetitions. All statistical analyses were performed by one-way ANOVA.

FIG. 18 shows the size distribution of extracellular vesicles (EVs) measured by Nanoparticle Tracking Analysis (NTA). (a) Naive SH-SY5Y cell-derived EVs, (b) miRNA-loaded SH-SY5Y cell-derived EVs, (c) Comparative EV diameter metrics (mean±SD). D10/D50/D90: EV diameters below which 10%/50%/90% of the population resides.

FIG. 19 shows a representative image combined by bright field image for droplets and fluorescence image for 4 different kinds of test samples: PBS (as a blank), HeLa cell EV (negative control), SH-SY5Y cell EV (normal), and SH-SY5Y cell EV±miRNA (abnormal).

FIG. 20 shows clustering analyses performed using dimensionality reduction on experimental data from four test sample groups—PBS (blank), HeLa (negative control), SH-SY5Y (normal), and SH-SY5Y±miRNA (abnormal)—for six different miRNAs relevant to neurodegenerative diseases.

FIG. 21 shows histograms of mean fluorescence intensity. The intensity distributions were plotted for each sample type and miRNA.

FIG. 22 shows quantification of EV miRNAs for each test sample: PBS (blank), HeLa EV (negative), SH-SY5Y EV (normal), and SH-SY5Y±miRNA (abnormal).

### DETAILED DESCRIPTION OF THE INVENTION AND PREFERRED EMBODIMENTS OF THE INVENTION

Unless otherwise defined, all technical and scientific terms used herein have the same meanings as typically understood by those skilled in the art to which the present invention belongs. In general, the nomenclature used herein is well known in the art and is typical.

Investigating protein-free fusion mechanisms in individual extracellular vesicle (EV) and liposome would not only improve understanding of various EV populations, but also allow for the investigation of RNA of single EVs, which may identify distinct subpopulations in disease condition. However, these analytical techniques require EV lysis, which in most cases results in reduced sensitivity, high cost, and labor intensiveness, time-consuming processes. The present inventors developed an approach for detection of individual EV subpopulations based on charge-mediated fusion of EVs and liposomes. The surface charge of liposomes was tuned by changing the ratio of positively and negatively charged lipids, and certain ratios exhibited very efficient and stable fusion to exosomes without compromising membrane properties confirmed by membrane mixing assays. This method leveraging the advantages of high fusion rate, rapid applicability, and broad range of charged-liposome EV detection (EV-CLIP) demonstrates remarkable sensitivity and selectivity for EV-derived RNAs in a lysis-free manner using droplet-microfluidics. In addition, EV-CLIP allows to the digital detection of EGFR L858R and T790M mutations in plasma samples collected from 73 lung cancer patients (17 patients without mutation and 56 patients with mutation) and 10 healthy donors without EV pre-isolation step, further simplifying the detection process and preventing EV loss. Overall, EV-CLIP holds great promise in the clinical setting for accurate quantification of rare EV subpopulations and provides novel opportunities to explore fundamental questions in cancer biology.

### Definitions

As used herein, the term “about” in reference to a number is generally taken to include numbers that fall within a range of 1%-10% in either direction (greater than or less than) of the number unless otherwise stated or otherwise evident from the context.

The terms “complement”, “complementary” or “complementarity” as used herein with reference to polynucleotides (i.e., a sequence of nucleotides such as an oligonucleotide or a target nucleic acid) refer to the Watson/Crick base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3” is complementary to the sequence “3′-T-C-A-5′.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

The term “substantially complementary” as used herein means that two sequences hybridize under stringent hybridization conditions. The skilled artisan will understand that substantially complementary sequences need not hybridize along their entire length. In particular, substantially complementary sequences may comprise a contiguous sequence of bases that do not hybridize to a target sequence, positioned 3′ or 5′ to a contiguous sequence of bases that hybridize under stringent hybridization conditions to a target sequence.

As used herein, the term “detecting” refers to determining the presence of a target nucleic acid in the sample. Detection does not require the method to provide 100% sensitivity and/or 100% specificity.

As used herein, the term “extracellular vesicle (EV)” refers to a small secretory vesicle (typically about 30-800 nm) that may comprise nucleic acids, proteins, or other biomolecules, and EVs include exosomes and microvesicles. EVs may act as cellular messengers by transporting biomolecules to various locations in living organisms or biological systems.

As used herein, the term “cancer cell-specific molecular beacon” refers to a molecular beacon that specifically binds to a cancer-specific substance that is present in cancer cell-derived extracellular vesicles but not in normal cell-derived extracellular vesicles.

The term “fluorophore” as used herein refers to a molecule that absorbs light at a particular wavelength (excitation frequency) and subsequently emits light of a longer wavelength (emission frequency). The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tm) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the terms “individual”, “patient”, or “subject” can be an individual organism, a vertebrate, a mammal, or a human. In a preferred embodiment, the individual, patient or subject is a human.

The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e., collisional) quenching or by Förster or fluorescence resonance energy transfer (“FRET”). Quenching by FRET is generally used in TaqMan® probes while proximal quenching is used in molecular beacon and Scorpion™ type probes.

As used herein, the “molecular beacon (MB)” is an oligonucleotide that forms a hairpin-shaped secondary structure with the 3′ end tagged with a quencher material, and the molecular beacon probe specifically hybridizes in a region complementary to a template gene during the annealing process, and the distance between the fluorescent material and the quencher material increases, thereby releasing inhibition of luminescence by the quencher material and fluorescing. On the other hand, unhybridized molecular beacons maintain the secondary structure thereof and are therefore inhibited by the quencher and do not fluoresce.

In an embodiment of the present invention, molecular beacons represented by the nucleotide sequences of SEQ ID NO: 1 and SEQ ID NO: 2 were respectively used to detect EGFR L858R and T790M mutations in H1975 lung cancer cell lines, and tumor-derived extracellular vesicles (tEVs) were detected using a molecular beacon represented by the nucleotide sequence of SEQ ID NO: 3, which is designed to target microRNA-21 (or miR-21) known to be upregulated in various tumor types such as breast cancer, colon cancer, lung cancer, pancreatic cancer, prostate cancer, and stomach cancer. In other embodiments, the molecular beacons comprise a nucleic acid sequence of SEQ ID NOs: 4-9.

As used herein, the term “lipid” or “lipid analogue” refers to a molecule including at least one hydrophobic moiety or group and optionally also at least one hydrophilic moiety or group. A molecule including hydrophobic and hydrophilic moieties is also often referred to as an amphipathic substance. Lipids are generally not very soluble in water. In an aqueous environment, the amphipathic property allows the molecules to self-assemble into organized structures and different phases. One of these phases, when they exist in vesicles, multilamellar/unilamellar liposomes or membranes in aqueous environments, is composed of a lipid bilayer. Hydrophobic groups include nonpolar groups including, but not limited to, long-chain saturated and unsaturated aliphatic hydrocarbon groups and such groups substituted with at least one aromatic, alicyclic or heterocyclic group(s). Hydrophilic groups may include polar and/or charged groups and may include carbohydrate, phosphate, carboxyl, sulfate, amino, sulfhydryl, nitro, hydroxyl, and other similar groups.

As used herein, the term “amphipathic” molecule refers to a molecule having both polar and nonpolar portions. An amphipathic compound often has a polar head attached to a long hydrophobic tail. In some embodiments, the polar portion is soluble in water, while the nonpolar portion is insoluble in water. Additionally, the polar portion may have a formal positive charge or a formal negative charge. Alternatively, the polar portion may have both formal positive and negative charges and may be a zwitterion or an internal salt. For the purposes of this specification, an amphipathic compound may be one or more natural or non-natural lipids and lipid-like compounds, but not limited thereto.

The term “lipid analogue”, “lipid-like compound”, or “lipid-like molecule” refers to a substance that is structurally and/or functionally lipid-like but may not be considered a lipid in the strict sense. For example, this term includes compounds capable of forming amphipathic layers when present in vesicles, multilamellar/unilamellar liposomes or membranes in aqueous environments, and includes surfactants or synthetic compounds having both hydrophilic and hydrophobic moieties. Generally, this term means a molecule including hydrophilic and hydrophobic moieties with different structural organizations that may or may not be similar to that of lipids. Herein, the term “lipid” should be construed to encompass both lipids and lipid analogues, unless it is clearly contradictory in context.

As used herein, the term “cationic lipid” or “cationic lipid analogue” refers to a lipid or lipid-like substance having a net positive charge. A cationic lipid or lipid analogue binds to a negatively charged nucleic acid by electrostatic interaction. In general, a cationic lipid has a lipophilic moiety such as a sterol, acyl chain, diacyl or larger acyl chain, and the head group of the lipid is usually positively charged.

As used herein, the term “anionic lipid” refers to any lipid that is negatively charged at a selected pH. As used herein, the term “neutral lipid” refers to any of a number of lipid species present in either an uncharged or neutral zwitterion form at a selected pH.

As used herein, the term “biological sample” includes a biological fluid sample and a biological tissue sample.

As used herein, the term “biological fluid” refers to any fluid isolated from or derived from organisms, including prokaryotes, eukaryotes, bacteria, fungi, yeast, invertebrates, vertebrates, reptiles, fish, insects, plants, and animals, and may include, but is not limited to, serum, plasma, whole blood, urine, saliva, breast milk, tears, sweat, synovial fluid, cerebrospinal fluid, semen, vaginal fluid, sputum, pleural effusion, lymph, ascites, and amniotic fluid. Bronchial washing fluid and culture fluid obtained from cultured cells (e.g., cell culture supernatant, conditioned culture fluid, cell culture fluid, or cell culture medium) may also be biological fluids.

As used herein, the term “biological tissue” refers to a collection of cells derived from prokaryotes, eukaryotes, bacteria, fungi, yeast, invertebrates, vertebrates, reptiles, fish, insects, plants, or animals. In addition, cultured cells may be biological tissues. Non-limiting examples of the biological tissue sample include surgical samples, biopsy samples, tissues, stool, plant tissue, insect tissue, and cultured cells.

### Detection of a Cancer Cell-Derived EV

Accordingly, an aspect of the present invention relates to a liposome for detecting a cancer cell-derived extracellular vesicle (EV) comprising a cationic lipid and a neutral lipid, in which a cancer cell-specific molecular beacon is encapsulated within the liposome.

In the present invention, the cancer-specific substance present in cancer cell-derived extracellular vesicles may include microRNA-21 (miR-21), microRNA-25 (miR-25), microRNA-27 (miR-27), microRNA-54 (miR-54), microRNA-155 (miR-155), microRNA-210 (miR-210), microRNA-375 (miR-375), microRNA-451 (miR-451), microRNA-486 (miR-486), microRNA-495 (miR-495), microRNA-574-3p (miR-574-3p), EGFR L858R mutation, EGFR T790M mutation, GPC1, KRAS, AR-V7, survivin, TK-1, c-Myc, GalNAc-T, Cyclin D1, and so on (Wei Pan, et al., Anal. Chem. 2013, 85, 21, 10581-10588; Xiang-Hong Peng, et al., Cancer Res (2005) 65 (5): 1909-1917), but is not limited thereto.

In certain embodiments, the cationic lipid or lipid analogue has a net positive charge only at a certain pH, particularly acidic pH, whereas, preferably, at a higher pH, such as physiological pH, it does not have any net positive charge and is uncharged, namely neutral. This ionizable behavior is deemed to enhance efficacy by aiding endosomal escape and reducing toxicity compared to particles that remain cationic at physiological pH.

In the present invention, the cationic lipid is selected from the group consisting of, but is not limited to, 1,2-dioleoyl-3-trimethylammonium propane (DOTAP), 1,2-dioleyloxy-3-dimethylamino-propane (DODMA), 1,2-di-O-octadecenyl-3-trimethylammonium propane (DOTMA), 3β-[N-(N′,N′-dimethylaminoethane) carbamoyl]cholesterol (DC-Chol), dimethyldioctadecylammonium bromide (DODAB), 1,2-dioleoyl-3-dimethylammonium propane (DODAP), dioctadecyldimethyl ammonium chloride (DODAC), 1,2-distearyloxy-N,N-dimethyl-3-aminopropane (DSDMA), 1,2-dimyristyloxy-propyl-3-dimethyl-hydroxyethyl ammonium bromide (DMRIE), 1,2-dimyristoyl-sn-glycero-3-ethylphosphocholine (DMEPC), 1,2-dimyristoyl-3-trimethylammonium propane (DMTAP), 1,2-diolcoyl-3-dimethyl-hydroxyethyl ammonium bromide (DORIE), 2,3-dioleyloxy-N-[2-(sperminccarboxamido) ethyl]-N,N-dimethyl-1-propanaminium (DOSPA), 1,2-dilinoleyloxy-N,N-dimethyl-3-aminopropane (DLinDMA), 1,2-dilinolenyloxy-N,N-dimethyl-3-aminopropane (DLenDMA), dioctadecylamidoglycylspermine (DOGS), 3-dimethylamino-2-(cholest-5-en-3-β-oxybutan-4-oxy)-1-(cis,cis-9,12-octadecadienoxy) propane (CLinDMA), 2-[5′-(cholest-5-en-3-β-oxy)-3′-oxapentoxy]-3-dimethyl-1-(cis,cis-9′,12′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-dilinoleoyloxy-N,N-dimethylpropylamine (DLinDAP), 1,2-N,N′-dilinoleylcarbamyl-3-dimethylaminopropane (DLincarbDAP), 1,2-dilinoleoylcarbamyl-3-dimethylaminopropane (DLinCDAP), 2,2-dilinoleyl-4-dimethylaminomethyl-[1,3]-dioxolane (DLin-KDMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino) butanoate (DLin-MC3-DMA), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), (±)-N-(2-aminoethyl)-N,N-dimethyl-2,3-bis(tetradecyloxy)-1-propanaminium bromide, BN-(4-carboxybenzyl)-N,N-dimethyl-2,3-bis(oleoyloxy) propan-1-aminium (DOBAQ), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido) ethyl]-3,4-di[oleyloxy]-benzamide (MVL5), 1,2-diolcoyl-sn-glycero-3-ethylphosphocholine (DOEPC), 2,3-bis(dodecyloxy)-N-(2-hydroxyethyl)-N,N-dimethylpropan-1-aminium bromide (DLRIE), N-(2-aminoethyl)-N,N-dimethyl-2,3-bis(tetradecyloxy) propan-1-aminium bromide (DMORIE), di((Z)-non-2-en-1-yl) 8,8′-((((2 (dimethylamino)ethyl) thio) carbonyl) azanediyl) dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), di((Z)-non-2-en-1-yl)-9-((4-(dimethylaminobutanoyl)oxy) heptadecanedioate (L319), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino) propionamide (lipidoid 98N12-5), and 1-[2-[bis(2-hydroxydodecyl)amino]ethyl-[2-[4-[2-[bis(2-hydroxydodecyl)amino]ethyl]piperazin-1-yl]ethyl]amino]dodecan-2-ol (lipidoid C12-200).

In the present invention, the liposome may comprise at least one non-cationic lipid such as anionic lipid and/or neutral lipid.

In the present invention, the neutral lipid is selected from the group consisting of, but is not limited to, 1,2-dioleoyl-sn-glycero-3-phosphocholine (DOPC), 1,2-dimyristoyl-sn-glycero-3-phosphocholine (DMPC), 1,2-diolcoyl-sn-glycero-3-phosphoethanolamine (DOPE), 1,2-dipalmitoyl-sn-glycero-3-phosphocholine (DPPC), 1,2-distearoyl-sn-glycero-3-phosphatidylserine (PS), phosphatidylethanolamine (PE), phosphatidylglycerol (PG), phosphoric acid (PA), and phosphatidylcholine (PC).

Preferably, the cationic lipid is 1,2-dioleoyl-3-trimethylammonium propane (DOTAP), and the neutral lipid is 1,2-dioleoyl-sn-glycero-3-phosphocholine (DOPC), but the present invention is not limited thereto.

In the present invention, the molar ratio (%) of the cationic lipid in the liposome may be 25 to 75%, but is not limited thereto.

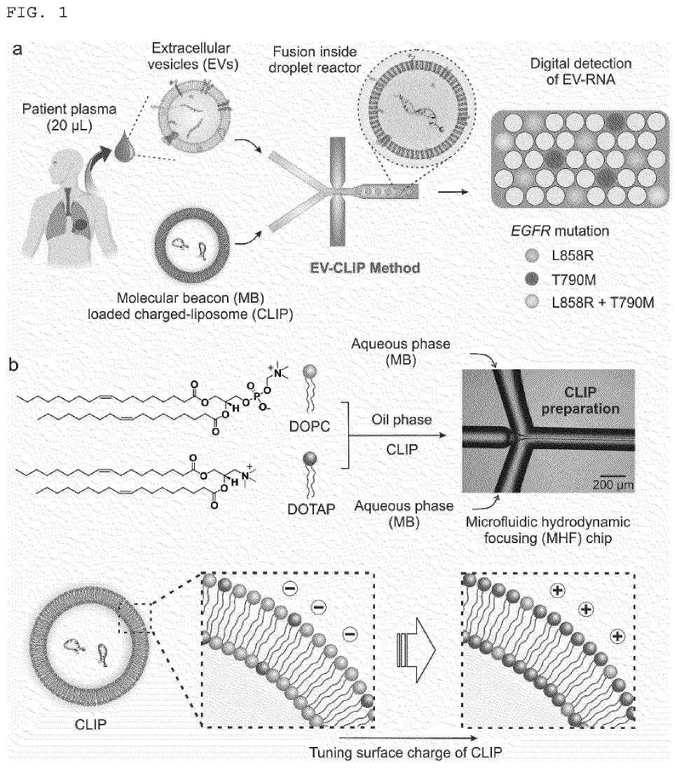

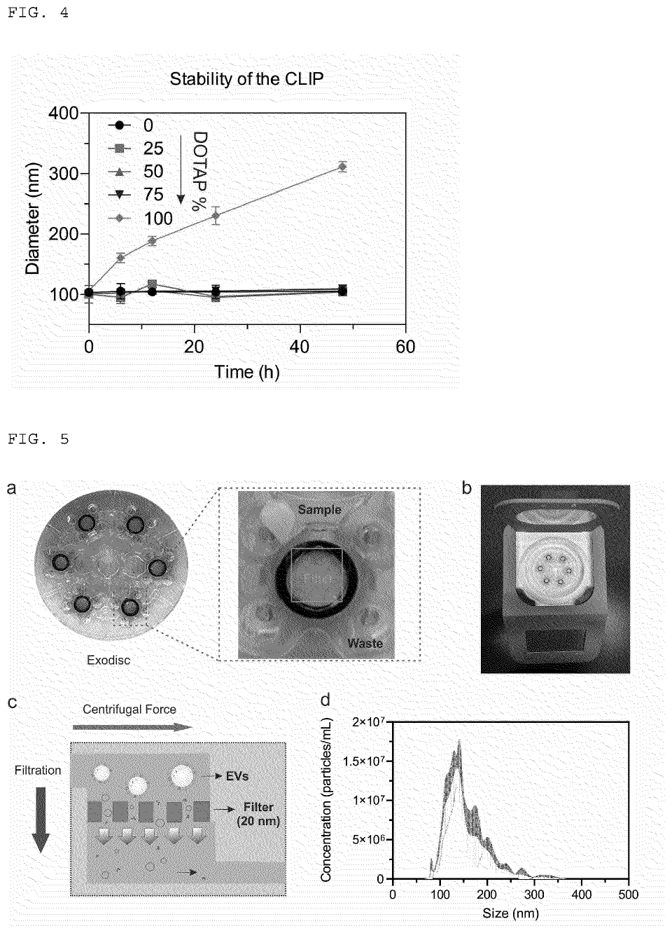

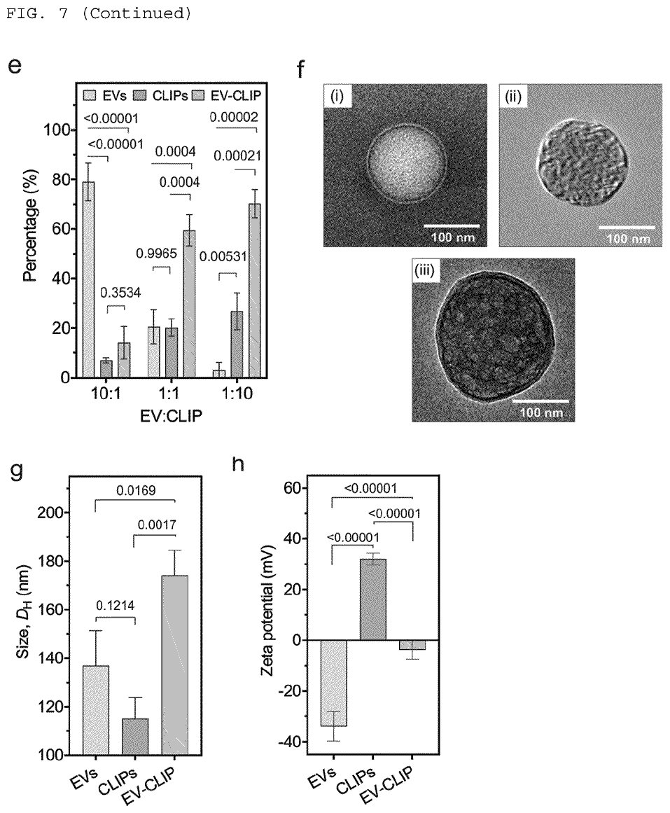

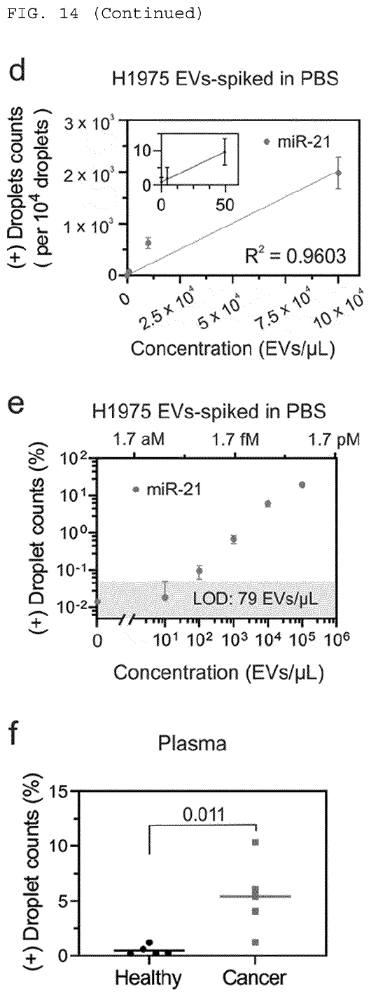

In an embodiment of the present invention, when 25% DOTAP was included in the total lipid, the surface charge shifted significantly from negative to positive, and the surface charge was maintained as positive up to 100% (FIG. 1d), and the fusion rate with extracellular vesicles was increased (FIG. 7c). However, the CLIP comprising 100% DOTAP showed instability and formed aggregates over time despite having the highest fusion efficiency (FIGS. 4 and 12). Therefore, the molar ratio (%) of the cationic lipid in the liposome is preferably 75%, and the molar ratio of cationic lipid to neutral lipid is 75:25, but the present invention is not limited thereto.

Another aspect of the present invention relates to a composition for detecting a cancer cell-derived extracellular vesicle comprising the liposome for detecting an extracellular vesicle.

Still another aspect of the present invention relates to a kit for detecting a cancer cell-derived extracellular vesicle comprising the liposome for detecting an extracellular vesicle.

Yet another aspect of the present invention relates to a method of detecting a cancer cell-derived extracellular vesicle comprising fusing the liposome for detecting an extracellular vesicle with an extracellular vesicle derived from a biological sample; and determining that the extracellular vesicle is a cancer cell-derived extracellular vesicle when a fluorescence signal is generated.

In the present invention, the fusion may be performed within a droplet reactor, but the present invention is not limited thereto.

In the present invention, the droplet reactor may comprise two aqueous phase channels, one oil phase channel, one junction, and one outlet channel, and may be characterized in that aqueous droplets are generated at the junction by introducing liposomes and extracellular vesicles derived from a biological sample respectively into the two aqueous phase channels, but the present invention is not limited thereto.

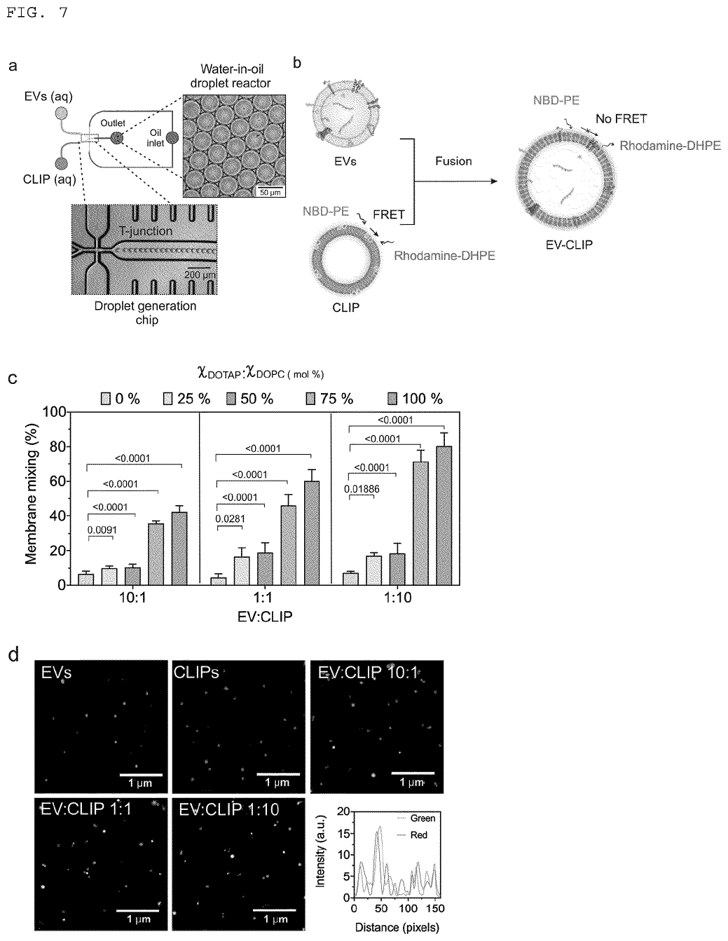

In an embodiment of the present invention, to ensure precise control over stoichiometry of vesicles (EVs and CLIPs) and prevent undesired aggregation, the entire fusion process was performed within the droplet reactor using a u Encapsulator that allows precise control, minimizing potential aggregation that may occur in bulk-scale reaction (FIG. 6). The u Encapsulator operates by allowing EVs and CLIPs as separate phases to flow through two aqueous inlets at a flow rate of 1.5 μl/min, while the continuous oil phase inlet delivers FC-40 as a biocompatible surfactant at a flow rate of 35 μl/min, enabling generation of aqueous droplets comprising both vesicles when these two vesicles meet at the junction within the oil phase (FIG. 7a).

In the present invention, the molar ratio of liposomes to extracellular vesicles derived from a biological sample may be 1:1 to 10:1, but is not limited to.

In an embodiment of the present invention, when the molar ratio of extracellular vesicles to liposomes was 10:1, the lipid membrane mixing percentage was 35.67±1.35%, when the molar ratio thereof was 1:1, the percentage was 45.91±6.33%, and when the molar ratio thereof was 1:10, the percentage was 71.45±6.58%. Therefore, the molar ratio of extracellular vesicles to liposomes is preferably 1:10, but is not limited thereto.

In an embodiment of the present invention, EGFR L858R and T790M mutations could be digitally detected without EV pre-isolation step via charged-liposome EV detection (EV-CLIP) in plasma samples collected from lung cancer patients and healthy donors, and EV loss was minimized due to simplification of the detection process. By fusing the liposome according to the present invention with the sample-derived EV and determining whether a fluorescence signal is generated, it is possible to easily determine whether the sample is a cancer cell.

A further aspect of the present invention relates to a composition for diagnosing cancer comprising the liposome for detecting an extracellular vesicle.

Still a further aspect of the present invention relates to a kit for diagnosing cancer comprising the liposome for detecting an extracellular vesicle.

Yet a further aspect of the present invention relates to a method of providing information for cancer diagnosis and a method of diagnosing cancer, comprising fusing the liposome for detecting an extracellular vesicle with an extracellular vesicle derived from a biological sample; and determining that the sample is cancerous when a fluorescence signal is generated.

In the present invention, the fusion may be performed within a droplet reactor comprising two aqueous phase channels, one oil phase channel, one junction, and one outlet channel, but the present invention is not limited thereto.

In an embodiment of the present invention, EGFR L858R and T790M mutations could be digitally detected without EV pre-isolation step via charged-liposome EV detection (EV-CLIP) in plasma samples collected from lung cancer patients and healthy donors. By fusing the liposome according to the present invention with the sample-derived EV and determining whether a fluorescence signal is generated, it is possible to easily determine whether the sample is cancerous. Even in early-stage cancer, mutations may be detected at high levels in a short time without preprocessing, making it easy to distinguish cancer patients from healthy donors, and thus demonstrating the value thereof over conventional methods in converting miRNA from tumor-derived EVs into standard clinical markers for cancer diagnosis.

In the present invention, the cancer may be selected from the group consisting of, but is not limited to, squamous cell carcinoma, small cell lung cancer, non-small cell lung cancer, lung cancer, peritoneal cancer, colon cancer, bile duct tumor, nasopharyngeal cancer, laryngeal cancer, bronchial cancer, thyroid cancer, oral cancer, osteosarcoma, gallbladder cancer, bile duct cancer, kidney cancer, bladder cancer, renal cell cancer, melanoma, brain cancer, glioma, glioblastoma, brain tumor, skin cancer, pancreatic cancer, breast cancer, liver cancer, bone marrow cancer, small intestine cancer, esophageal cancer, large intestine cancer, stomach cancer, eye cancer, urethral cancer, cervical cancer, prostate cancer, ovarian cancer, metastatic cancer, head and neck cancer, rectal cancer, non-Hodgkin's lymphoma, multiple myeloma, acute myelogenous leukemia, lymphoma, acute lymphoblastic leukemia, and chronic myelogenous leukemia.

In the present invention, the diagnostic kit may additionally comprise various components such as one or more types of other component compositions, solutions or devices suitable for the diagnostic method. The diagnostic kit may additionally comprise a compartmented carrier means containing a biological sample, a container containing a reagent, etc. The carrier means is suitable for containing one or more containers, such as bottles or tubes, each container containing independent components used in the method of the present invention. Herein, the required formulation may be easily dispensed into a container by those skilled in the art.

### Detection of a Pathological Cell-Derived EV in Neurodegenerative Disease

In one aspect, the present disclosure provides a liposome for detecting a pathological cell-derived extracellular vesicle (EV) from a subject with a neurodegenerative disease comprising a cationic lipid and a neutral lipid, in which a pathological cell-specific molecular beacon is encapsulated within the liposome. In some embodiments, the pathological cell is a cell (e.g., a neuron) that is affected by the neurodegenerative disease. In some embodiments, the neurodegenerative disease is selected from the group consisting of Huntington's disease, Alzheimer's disease, and Parkinson's disease. In some embodiments, the molecular beacon comprises a nucleic acid and a fluorophore. In some embodiments, the nucleic acid comprises a sequence encoding one or more of SEQ ID NOs: 4-9.

In some embodiments, the cationic lipid is selected from the group consisting of 1,2-dioleoyl-3-trimethylammonium propane (DOTAP), 1,2-dioleyloxy-3-dimethylamino-propane (DODMA), 1,2-di-O-octadecenyl-3-trimethylammonium propane (DOTMA), 3B-[N-(N′,N′-dimethylaminoethane)-carbamoyl]cholesterol (DC-Chol), dimethyldioctadecylammonium bromide (DODAB), 1,2-dioleoyl-3-dimethylammonium-propane (DODAP), dioctadecyldimethyl ammonium chloride (DODAC), 1,2-distearyloxy-N,N-dimethyl-3-aminopropane (DSDMA), 1,2-dimyristyloxy-propyl-3-dimethyl-hydroxyethyl ammonium bromide (DMRIE), 1,2-dimyristoyl-sn-glycero-3-ethylphosphocholine (DMEPC), 1,2-dimyristoyl-3-trimethylammonium propane (DMTAP), 1,2-diolcoyl-3-dimethyl-hydroxyethyl ammonium bromide (DORIE), 2,3-dioleyloxy-N-[2-(sperminecarboxamido) ethyl]-N,N-dimethyl-1-propanaminium (DOSPA), 1,2-dilinoleyloxy-N,N-dimethyl-3-aminopropane (DLinDMA), 1,2-dilinolenyloxy-N,N-dimethyl-3-aminopropane (DLenDMA), dioctadecylamidoglycylspermine (DOGS), 3-dimethylamino-2-(cholest-5-en-3-β-oxybutan-4-oxy)-1-(cis,cis-9, 12-octadecadienoxy) propane (CLinDMA), 2-[5′-(cholest-5-en-3-β-oxy)-3′-oxapentoxy]-3-dimethyl-1-(cis,cis-9′,12′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-dilinoleoyloxy-N,N-dimethylpropylamine (DLinDAP), 1,2-N,N′-dilinoleylcarbamyl-3-dimethylaminopropane (DLincarbDAP), 1,2-dilinoleoylcarbamyl-3-dimethylaminopropane (DLinCDAP), 2,2-dilinoleyl-4-dimethylaminomethyl-[1,3]-dioxolane (DLin-KDMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino) butanoate (DLin-MC3-DMA), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), (±)-N-(2-aminoethyl)-N,N-dimethyl-2,3-bis(tetradecyloxy)-1-propanaminium bromide (BAE-DMRIE), N-(4-carboxybenzyl)-N,N-dimethyl-2,3-bis(oleoyloxy) propan-1-aminium (DOBAQ), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido) ethyl]-3,4-di[alkyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-aminium bromide (DLRIE), N-(2-aminoethyl)-N,N-dimethyl-2,3-bis(tetradecyloxy) propan-1-aminium bromide (DMORIE), di((Z)-non-2-en-1-yl) 8,8′-((((2 (dimethylamino)ethyl) thio) carbonyl) azanediyl) dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), di((Z)-non-2-en-1-yl)-9-((4-(dimethylaminobutanoyl)oxy) heptadecanedioate (L319), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino) propionamide (lipidoid 98N12-5), and 1-[2-[bis(2-hydroxydodecyl)amino]ethyl-[2-[4-[2-[bis(2-hydroxydodecyl)amino]ethyl]piperazin-1-yl]ethyl]amino]dodecan-2-ol (lipidoid C12-200). In some embodiments, the liposome comprises multiple cationic lipids as described herein.

In some embodiments, the neutral lipid is selected from the group consisting of 1,2-dioleoyl-sn-glycero-3-phosphocholine (DOPC), 1,2-dimyristoyl-sn-glycero-3-phosphocholine (DMPC), 1,2-diolcoyl-sn-glycero-3-phosphoethanolamine (DOPE), 1,2-dipalmitoyl-sn-glycero-3-phosphocholine (DPPC), 1,2-distearoyl-sn-glycero-3-phosphatidylserine (PS), phosphatidylethanolamine (PE), phosphatidylglycerol (PG), phosphoric acid (PA), and phosphatidylcholine (PC). In some embodiments, the liposome comprises multiple neutral lipids as described herein.

In some embodiments, the cationic lipid is 1,2-dioleoyl-3-trimethylammonium propane (DOTAP) and the neutral lipid is 1,2-dioleoyl-sn-glycero-3-phosphocholine (DOPC). In some embodiments, the liposome comprises a molar ratio (%) of the cationic lipid in the liposome that is 25 to 75%.

In another aspect, the present disclosure provides a composition for detecting a pathological cell-derived extracellular vesicle comprising the liposome of any of the preceding embodiments.

In another aspect, the present disclosure provides a kit for detecting a pathological cell-derived extracellular vesicle comprising the liposome according to any of the preceding embodiments.

In another aspect, the present disclosure provides a method of detecting a pathological cell-derived extracellular vesicle, comprising: fusing the liposome according to any of the preceding embodiments with an extracellular vesicle derived from a biological sample; and determining that the extracellular vesicle is a pathological cell-derived extracellular vesicle when a fluorescence signal is generated. In some embodiments, the fusing is performed within a droplet reactor comprising two aqueous phase channels, one oil phase channel, one junction, and one outlet channel. In some embodiments, aqueous droplets are generated at the junction by introducing the liposome and the extracellular vesicle derived from the biological sample respectively into the two aqueous phase channels.

In another aspect, the present disclosure provides a diagnostic composition comprising the liposome according to any of the preceding embodiments.

In another aspect, the present disclosure provides a kit comprising the liposome according to any of the preceding embodiments and instructions for use thereof. In some embodiments, the instructions for use comprise diagnosing a neurodegenerative disease selected from the group consisting of Huntington's disease, Alzheimer's disease, and Parkinson's disease.

In another aspect, the present disclosure provides a method of selecting a subject having neurodegenerative disease for treatment, comprising: fusing the liposome according to any of the preceding embodiments with an extracellular vesicle derived from a biological sample; and selecting the subject for treatment when a fluorescence signal is generated. In some embodiments, the fusing is performed within a droplet reactor comprising two aqueous phase channels, one oil phase channel, one junction, and one outlet channel. In some embodiments, the neurodegenerative disease is selected from the group consisting of Huntington's disease, Alzheimer's disease, and Parkinson's disease.

### Suitable Molecular Beacon Fluorophores and Quenchers

The molecular beacons of the present disclosure comprise a detectable moiety. In some embodiments, the detectable moiety or label is a fluorophore. Suitable fluorescent moieties include, but are not limited to the following fluorophores: cyanine 3 fluorophore (Cy3), cyanine 5 fluorophore (Cy5), 4-acetamido-4′-isothiocyanatostilbene-2,2′disulfonic acid, acridine and derivatives (acridine, acridine isothiocyanate), Alexa Fluors (Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes)), 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl) maleimide, anthranilamide, BODIPY® R-6G, BOPIPY® 530/550, BODIPY® FL, Brilliant Yellow, Cal Fluor Red 610® (CFR610), coumarin and derivates thereof. In some embodiments, the fluorophore is Cy3.

Suitable quenchers are selected based on the fluorescence spectrum of the particular fluorophore. Useful quenchers include, for example, the Black Hole™ quenchers BHQ1, BHQ2, and BHQ3 (Biosearch Technologies, Inc.), and the ATTO-series of quenchers (ATTO 540Q, ATTO 580Q, and ATTO 612Q; Atto-Tec GmbH). In some embodiments, the quencher is BHQ2.

### Experimental Examples

A better understanding of the present invention may be obtained through the following examples. These examples are merely set forth to illustrate the present invention and are not to be construed as limiting the scope of the present invention, as will be apparent to those skilled in the art.

### Example 1: Materials and Methods

### Example 1-1: Reagents and Equipment

0.2 μm syringe filter (Minisart® NML Syringe Filters, S6534), 1× phosphate-buffered saline (pH 7.4, Gibco), antibiotic/antimycotic (Gibco, 15240062), anti-CD63 (BD Bioscience, BD556019), anti-CD81 (BD Bioscience, BD555675), anti-CD9 (BD Bioscience, BD555370), anti-EGFR (Abcam, ab192263), EV-depleted FBS (Systems Biosciences Inc), Exodisc™-C (LabSpinner), molecular beacon (Oligo, Macrogen), NBD-PE (N-(7-nitrobenz-2-oxa-1,3-diazol-4-yl)-1,2-dihexadecanoyl-sn-glycero-3-phosphoethanolamine, triethylammonium salt, ThermoFisher, N360), Rhodamine-DHPE (Lissamine rhodamine B 1,2-dihexadecanoyl-sn-glycero-3-phosphoethanolamine, triethylammonium salt, ThermoFisher, L1392), and TMB solution (Sigma-Aldrich, T0440-100ML).

0.2 μm syringe filter (Minisart® CA Syringe Filters, 16534K), u Encapsulator 2 reagent droplet chip (Dolomite Microfluidics 3200529), 100K Amicon centrifugal filter (Merck Millipore, UFC810096), 150 μm aqueous 5-input chip 3D (Dolomite Microfluidic 3200834), 30 μm chamber chip (Microfluidic ChipShop, 10001447), 96-well plate (Corning, 3364), confocal laser scanning microscope (Zeiss LSM 780NLO, Zeiss), Malvern Zetasizer (Nano ZS), NanoSight instrument (Nanosight NS500, Malvern Instruments), TECAN M2000 Pro Plate Reader (Tecan), and transmission electron microscope (JEOL, JEM-2100).

### Example 1-2: Cell Culture

H1975 cells (ATCC) were cultured at 37° C. in the presence of 5% CO₂ under static conditions in Dulbecco's Modified Eagle Medium (DMEM) (Gibco, 11965092) supplemented with 5% (v/v) FBS (Gibco) and 1% antibiotic/antimycotic (100 U/ml penicillin and 100 mg/ml streptomycin, Gibco, 15240062).

### Example 1-3: Isolation of Extracellular Vesicle from Cell Culture

Extracellular vesicles were isolated from cell culture medium using a commercialized Exo-Disc platform (Woo, H. K., et al., Acs Nano, 2017. 11 (2): p. 1360-1370). 70-80% confluent 10 cm culture dishes of cells were washed twice with 1× phosphate-buffered saline (pH 7.4, Gibco) and then cultured for 48 hours in respective medium supplemented with 5% EV-depleted FBS (Systems Biosciences Inc.) and 1% antibiotic/antimycotic, after which the cell culture supernatant was collected, centrifuged at 300 g for 10 minutes and then at 2,000 g for another 10 minutes to remove cell debris, and passed through a 0.2 μm syringe filter (Minisart® NML Syringe Filters, S6534). Thereafter, the clarified supernatant was concentrated using an ExoDisc platform, a centrifugal disc equipped with anodized aluminum oxide filters with a pore diameter of 0.02 μm (Exodisc™-C, LabSpinner). The supernatant was centrifuged at 3,000 rpm (approximately 500 g) and passed through the filter, and 100 μl of concentrated EVs from the collection chamber was resuspended in 1×PBS with a dilution factor of 2. Thereafter, the isolated EVs were analyzed using a NanoSight instrument and a Malvern Zetasizer to determine the concentration, size, and zeta potential distribution, and the EVs were aliquoted and stored at −80° C. until they were used for further experiments.

### Example 1-4: Liposome Synthesis

Liposomes were synthesized by microfluidic hydrodynamic focusing method using a commercialized platform (Dolomite Microfluidics). Lipid mixtures for liposomes were prepared by dissolving various ratios of 18:1 TAP or DOTAP (1,2-diolcoyl-3-trimethylammonium propane, Avanti 890890P) and 18:1 (49-Cis) PC or DOPC (1,2-diolcoyl-sn-glycero-3-phosphocholine, Avanti 850375P)-0%, 25%, 50%, 75%, and 100% DOTAP—in 1 ml of pure ethanol (Duksan Pure Chemicals, UN1170) filtered using a 0.2 μm syringe filter (Minisart® CA Syringe Filters, 16534K) to reach a final concentration of 5 mg/ml. CLIP synthesis was performed using a 150 μm hydrophilic 5-input chip 3D (Dolomite Microfluidic 3200834) by adjusting the flow rate of an aqueous phase (1X phosphate-buffered saline, pH 7.4, Gibco) to be 50 μl/min and the flow rate of an oil phase (lipid mixture) to be 5 μl/min. The suspension was collected in a 1.5 ml microcentrifuge tube for 15 minutes, purified using a 100K Amicon centrifugal filter (Merck Millipore, UFC810096) with twice PBS washing, and then analyzed using a NanoSight instrument (Nanosight NS500, Malvern Instruments) and a Malvern Zetasizer (Nano ZS) to determine the concentration, size, and zeta potential distribution. Thereafter, CLIPs were aliquoted and stored at 4° C. for further use.

### Example 1-5: Microfluidic Aided EV-CLIP Fusion

Microfluidic Aided fusion was performed using a commercialized platform (Dolomite Microfluidics). The glass chip (u Encapsulator 2 Reagent Droplet Chip, Dolomite Microfluidics 3200529) consists of four channels (two aqueous phase channels, one oil phase channel, and one outlet channel). Liposomes and extracellular vesicles at the same concentration in PBS were loaded into the aqueous channels of the microfluidic chip, and droplets with a diameter of 30 μm were generated in the FC-40 (RAN Biosciences, 008-FluoroSurfactant-2wtF-50G) oil phase. The numbers of extracellular vesicles and liposomes can be controlled by varying the initial sample input concentration in the range of 10 to 10⁵ particles/μl, and the pressure of the pumps were maintained at 2,500 mBar and 1,100 mBar for oil and aqueous phases, respectively. The droplets were collected in microcentrifuge tubes for 20 minutes and stored at 4° C. until they were used for further experiments.

### Example 1-6: Detection of Extracellular Vesicle Through Fusion with MB-CLIP

Detection of extracellular vesicles was performed using the microfluidic aided fusion method described above. Various concentrations of extracellular vesicles (10−10⁵ particles/μl) and a fixed concentration of molecular beacon containing CLIPs (miR-21 or EGFR L858R and T790M mutations) both in 1×PBS, were loaded into the aqueous channels of the microfluidic chip, where droplets of 30 μm in diameter were generated in the FC-40 oil phase. The pressures of the pumps were maintained at 2,500 mBar and 1,100 mBar in oil and aqueous phases, respectively. The droplet solution was collected in 1.5 ml brown microcentrifuge tubes for 20 minutes and stored at 4° C. until it is used for further experiments.

### Example 1-7: EV-CLIP Colocalization Experiment

For EV-liposome colocalization studies, EVs and liposomes were labeled with 3,3′-dioctadecyloxacarbocyanine perchlorate (DiO) dye (ThermoFisher Scientific, V22886) and Rhodamine-DHPE (Lissamine rhodamine B 1,2 dihexadecanoyl-sn-glycero-3-phosphoethanolamine, triethylammonium salt) (ThermoFisher, L1392), respectively. For EV labeling, 100 μl of pre-concentrated EVs from the ExoDisc platform were incubated with 2 μl of 200 nM DiO dye solution on a shaker at room temperature for 20 minutes. After incubation, EVs were centrifuged at 3,000 rpm and washed twice with IX PBS. The concentrated EVs from the chamber were resuspended in 1×PBS with a dilution factor of 2. For liposome labeling, 1 mol % Rhodamine-DHPE was incorporated into the lipid mixture. The volume was then adjusted to reach a final lipid concentration of 5 mg/ml. Rho-liposomes were synthesized by the microfluidic hydrodynamic focusing method described above. The synthesized liposomes were purified by washing with PBS using a 100K Amicon centrifugal filter to remove free dyes. The dye-modified liposomes were fused with extracellular vesicles using the microfluidic aided fusion method described above. The fused vesicles were then diluted with 1×PBS and imaged with a confocal laser scanning microscope (Zeiss LSM 780NLO) using a ×10 Plan-Apochromat and a 0.45 NA objective lens equipped with an LU-NV laser unit 488 nm (green/Alexa Fluor 488) and 560 nm (red/Cy5). The colocalization analysis was performed using Fiji.

### Example 1-8: NBD-PE Fusion Quantification Assay

Fusion quantification was performed using a published method (Murtas, G., Systems and synthetic biology, 2010. 4 (2): p. 85-93). 1 mol % of Rhodamine-DHPE (Lissamine rhodamine B 1,2 dihexadecanoyl-sn-glycero-3-phosphoethanolamine, triethylammonium salt) (ThermoFisher, L1392) and NBD-PE (N-(7-nitrobenz-2-oxa-1,3-diazol-4-yl)-1,2-dihexadecanoyl-sn-glycero-3-phosphoethanolamine, triethylammonium salt) (ThermoFisher, N360) were incorporated into the lipid mixture. Thereafter, the volume was adjusted to reach a final lipid concentration of 5 mg/ml. Rho-NBD liposomes were synthesized by the microfluidic hydrodynamic focusing method described above. The synthesized liposomes were purified by washing with PBS using a 100K Amicon centrifugal filter to remove free dyes. The dye-modified liposomes were fused with the extracellular vesicles using the microfluidic aided fusion method described above. The fluorescence signals from the fused vesicles were measured using a spectrophotometer (M2000 Pro, Tecan) with excitation-emission wavelengths of 485/535 nm, respectively. The fusion percentage was calculated using the following formula:

$$Fusion \% = \frac{F - {F}_{min}}{{F}_{max} - {F}_{min}} \times 100 \%$$

### Example 1-9: Loading of Molecular Beacon into the Liposomes

The miR-21, EGFR L858R, and EGFR T790M targeting molecular beacons (Oligo, Macrogen) were loaded into the liposomes by incorporating a certain amount of molecular beacons with a total concentration of 1 μM in 1×PBS. The liposomes were then synthesized using the microfluidic hydrodynamic focusing (MHF) method with a final lipid concentration of 5 mg/ml. The miR-21 targeting beacon was loaded into a batch of liposomes, whereas the EGFR L858R and T790M mutation-specific beacons were simultaneously loaded into a different batch of liposomes. The flow rates of the pumps were maintained at 50 μl/min and 5 μl/min for aqueous and oil phases, respectively. Thereafter, the molecular beacon-loaded liposomes were purified by washing twice with PBS using a 100K Amicon centrifugal filter, and then analyzed using a Nanosight instrument and a Malvern Zetasizer to determine the concentration, size, and zeta potential distribution. The liposomes were then stored at 4° C. for further use.

### Example 1-10: Droplet Imaging

For imaging, 6 μl of the droplet solution was loaded into a 30 μm chamber chip (Microfluidic ChipShop, 10001447) and the droplets were observed in a confocal laser scanning microscope (Zeiss LSM 780NLO) using a ×10 Plan-Apochromat and a 0.45 NA objective lens equipped with an LU-NV laser unit 488 nm (green/Alexa Fluor 488) and 560 nm (red/Cy5).

All images for droplet analysis were processed using Fiji.

### Example 1-11: Healthy Plasma Spiking Experiment

Healthy human blood plasma approved by the Institutional Review Board (IRB) was obtained from the Red Cross (UNISTIRB-19-41-C). Human blood plasma was diluted in 1×PBS with a dilution factor of 10 and then spiked with H1975 cell-derived EVs at different concentrations ranging from 10 to 10⁵ particles/μl. The spiked plasma samples and molecular beacon-loaded CLIPs (miR-21 or EGFR L858R and T790M mutations) were loaded into the droplet chip for microfluidic aided fusion. The pressure of the pumps were maintained at 2,500 mBar and 1,100 mBar for oil and aqueous phases, respectively. The droplets were collected in microcentrifuge tubes for 20 minutes and stored at 4° C. until they were used for further analysis.

### Example 1-12: EGFR Mutation Detection in Patient Sample

A total of 83 samples, including 10 healthy donors, 17 lung cancer samples without mutation (6 stage I, 3 stage II, 3 stage III, and 5 stage IV), and 56 lung cancer samples with EGFR mutation (18 stage I, 6 stage II, 7 stage III, and 25 stage IV), was obtained from Inha University Hospital (2019-11-017), Chonnam National University Hwasun Hospital (CNUHH-2022-021), and Pusan National University Hospital (2106-044-104): all healthy donor plasma samples were provided by Inha University Hospital; one stage II sample and one stage III sample without mutation, and 12 stage I, 5 stage II, 6 stage III, and 16 stage IV samples with EGFR mutation were provided by Chonnam National University Hwasun Hospital; and the remaining samples were provided by Pusan National University Hospital. 20 μl of the patient samples and the CLIPs containing molecular beacons detecting EGFR L858R and T790M were loaded into the droplet chip for microfluidic aided fusion. The pressures of the pumps were maintained at 2,500 mBar and 1,100 mBar for oil and aqueous phases, respectively. The droplets were collected in microcentrifuge tubes for 20 minutes and stored at 4° C. until they were used for further analysis.

### Example 1-13: Statistical Analysis

The experiments were performed independently at least three times, and the number of independent replicates used for each experiment is indicated in each figure legend. Comparisons between two groups were performed by a two-tailed unpaired Student's t-test, and comparisons among three or more groups were performed by one-way ANOVA. All statistical analyses were performed in Graphpad Prism 9.3.1.

### Example 2: High-Throughput Charge-Tuning of Liposomes

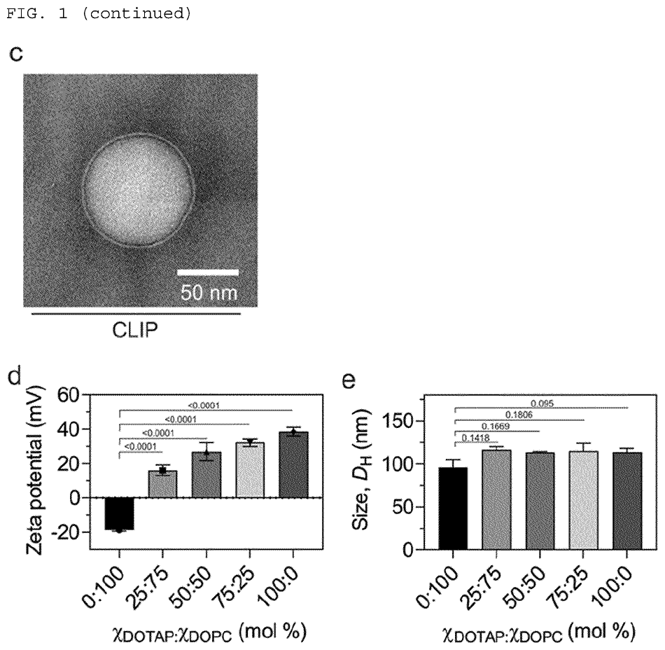

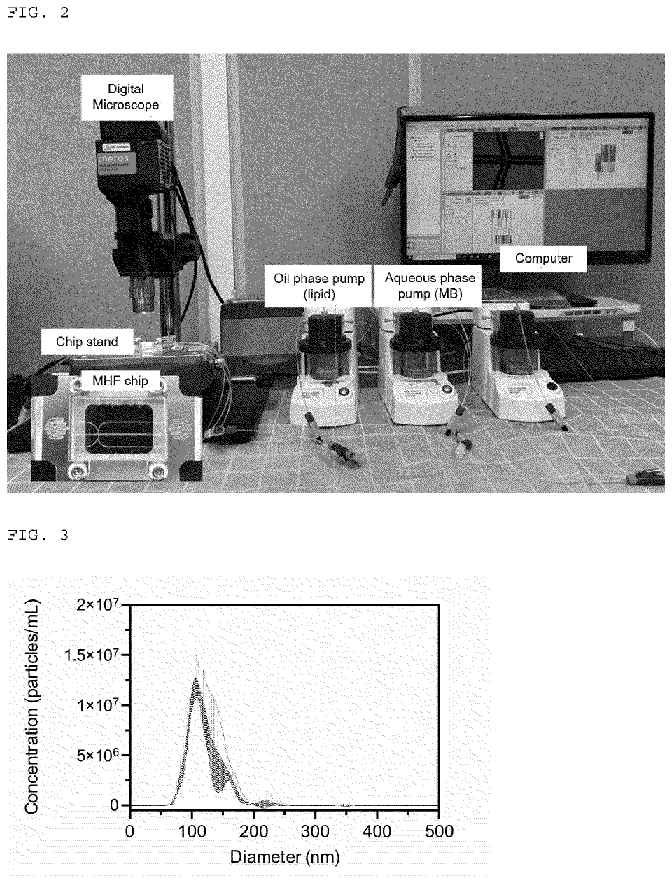

In the present invention, two types of lipids, cationic 1,2-dioleoyl-3-trimethylammonium propane (DOTAP) and zwitterionic 1,2-diolcoyl-sn-glycero-3-phosphocholine (DOPC), were utilized as building blocks for the preparation of charged liposomes (CLIPs) to achieve high-throughput charge-tuning of liposomes. By utilizing microfluidic hydrodynamic focusing (MHF) for liposome formulation (Jahn, A., et al., J Am Chem Soc 2004, 126 (9), 2674-5), variation in lipid composition were successfully achieved, thus efficiently and rapidly tuning the surface charge of CLIPs (FIGS. 1a, 1b, and 2). The microfluidic device facilitated the CLIP formation by allowing a lipid solution (composed of DOTAP and DOPC) dissolved at a concentration of 5 mg/ml in ethyl alcohol to flow through the central inlet channel at a rate of 5 μl/min, and an aqueous solution to flow at a rate of 50 μl/min through two lateral inlet channels, resulting in successful formation of CLIPs. FIG. 1c shows that the round morphology of the liposomes confirmed by a transmission electron microscope (TEM) (diameter: 92.5±5.65 nm) is consistent with the size measured via dynamic light scattering (DLS) and nanoparticle tracking analysis (NTA) (FIG. 3). As shown in FIG. 1d, the surface charge of CLIPs was tuned from −18.63±0.76 mV to ±38.67±2.51 mV by changing the composition of the building block lipids, DOTAP and DOPC. Ideally, the higher the ratio of DOTAP, the more positive the surface charge of CLIPS, and vice versa. By adjusting the ratio of DOTAP in the lipids from 0% (pure DOPC) to 100% (pure DOTAP), a noticeable shift in the surface charge from negative to positive was observed when 25% DOTAP was incorporated into the total lipid amount. This change was consistent with the predicted results, and the surface charge was found to increase proportionally with an increase in the ratio of DOTAP (FIG. 1d). In particular, after storing the liposomes for 48 hours, the size of the liposomes with 0%, 25%, 50%, and 75% DOTAP remained stable over time. However, the liposomes with 100% DOTAP continuously increased in size up to 300 nm, and specifically, showed about three times increase in size to 311.50±8.51 nm (FIGS. 1e and 4).

### Example 3: Charge-Induced Fusion of Liposomes

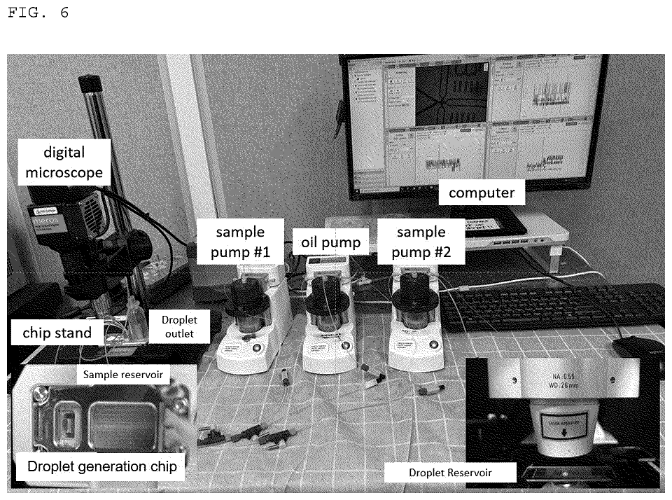

After fine-tuning the surface charge of liposomes, the effect on fusion of extracellular vesicles and CLIPs was determined. First, a rapid, label-free, and high-yield method was performed to isolate EVs from H1975 cells using a lab-on-a-disc system integrated with nanofilters (ExoDisc) (FIG. 5a), and EVs with a size of 20-200 nm could be concentrated within 30 minutes using cell culture supernatant (CCS) as a starting material in a tabletop-sized centrifugal microfluidic system (FIG. 5b). The homogeneous size distribution of EVs was confirmed by utilizing nanoparticle tracking analysis (NTA) and dynamic light scattering (DLS) (FIGS. 5c and 5d).

Next, to ensure precise control over stoichiometry of vesicles (EVs and CLIPs) and prevent undesired aggregation, the entire fusion process was conducted within a droplet reactor using a u Encapsulator that allows precise control, minimizing potential aggregation that could occur in bulk-scale reactions (FIG. 6). The uEncapsulator operates by allowing EVs and CLIPS as separate phases to flow through two aqueous inlets at a flow rate of 1.5 μl/min, while the continuous oil phase inlet delivers the biocompatible surfactant FC-40 at a flow rate of 35 μl/min, enabling the generation of aqueous droplets containing both vesicles when these two vesicles meet at the junction within the oil phase (FIG. 7a). The amounts of EVs and CLIPs contained in one droplet can be controlled by adjusting the initial input concentration of the vesicles according to the Poisson distribution calculation (Table 1).

TABLE 1
Poisson distribution calculation for EV
distribution in individual droplets

| | Average | | | | | |
|---|---|---|---|---|---|---|
| | # of | | | | | |
| Concentration | EV/ | Probability of a droplet containing (%) | | | | |
| (particles/ml) | droplet | 0 EV | 1 EV | 2 EVs | 3 EVs | 4 EVs |
| 10⁴ | 0.00007 | 99.993 | 0.007 | 0 | 0 | 0 |
| 10⁵ | 0.0007 | 99.93 | 0.07 | 0 | 0 | 0 |
| 10⁶ | 0.007 | 99 | 1 | 0 | 0 | 0 |
| 10⁷ | 0.07 | 93 | 7 | 0 | 0 | 0 |
| 10⁸ | 0.7 | 49 | 35 | 12 | 3 | 1 |

Table 1 describes the probability (%) of a droplet containing a certain number of EVs in the concentration range of 104 to 108 particles/ml.

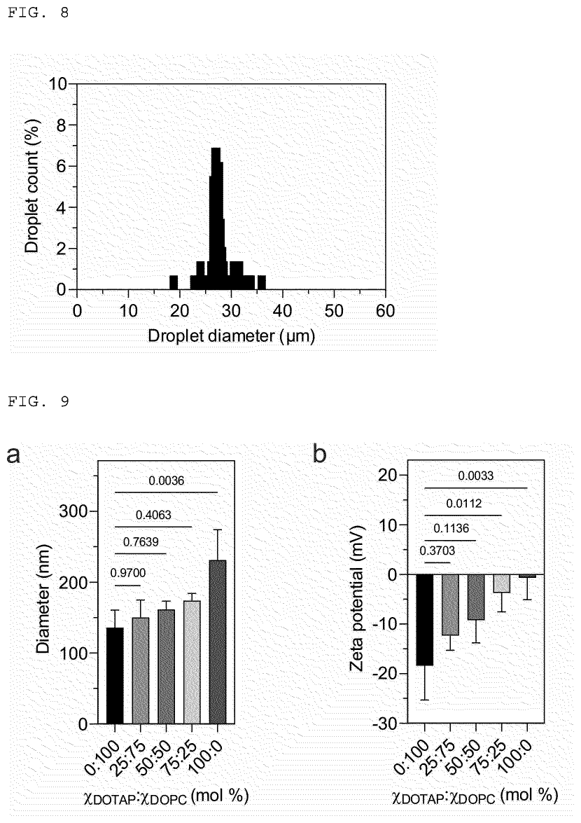

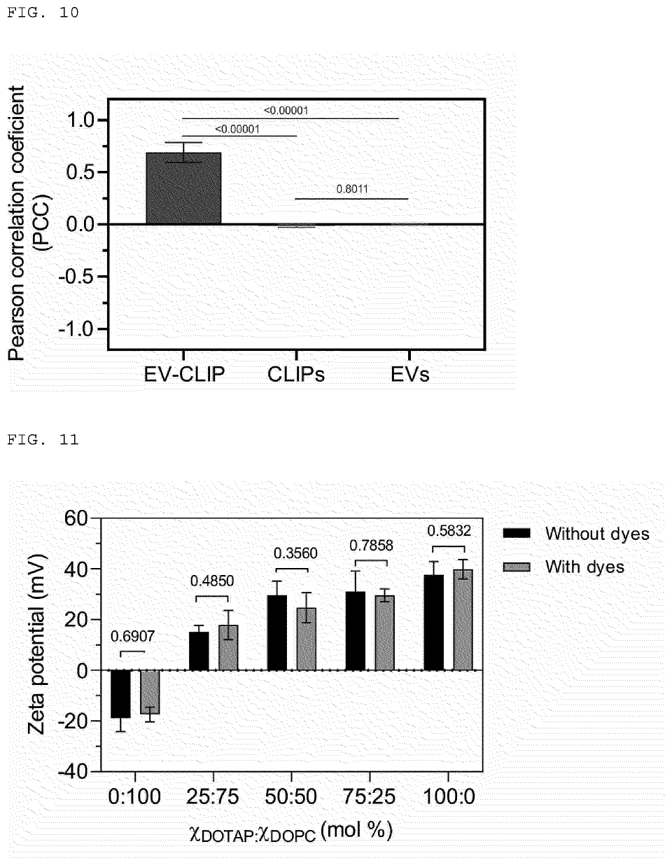

In this microfluidic setup, the initial input concentration of EVs (ranging from 10 to 10⁵ EVs/μl) and the ratio thereof with CLIPs were controlled within a single droplet having a size of 27.40±2.06 μm (FIG. 8). As shown in FIG. 7f, the regular morphology of individuals, semi-fused vesicles and fused vesicles was confirmed through TEM analysis. Additionally, DLS analysis showed that the size of the vesicle population increased after fusion, which is consistent with the observed results (FIG. 7g). However, when the ratio of DOTAP in CLIPS for EV fusion was gradually increased, the size of the fused vesicles remained unchanged until reaching 75% DOTAP, beyond which a significant increase in the vesicle size was observed (FIG. 9a). The confirmation of vesicle fusion was further supported by observing the changes in the surface charge of the fused vesicles using a Zeta sizer, and increased zeta potential correlated with a higher DOTAP ratio, validating the occurrence of fusion (FIG. 9b).

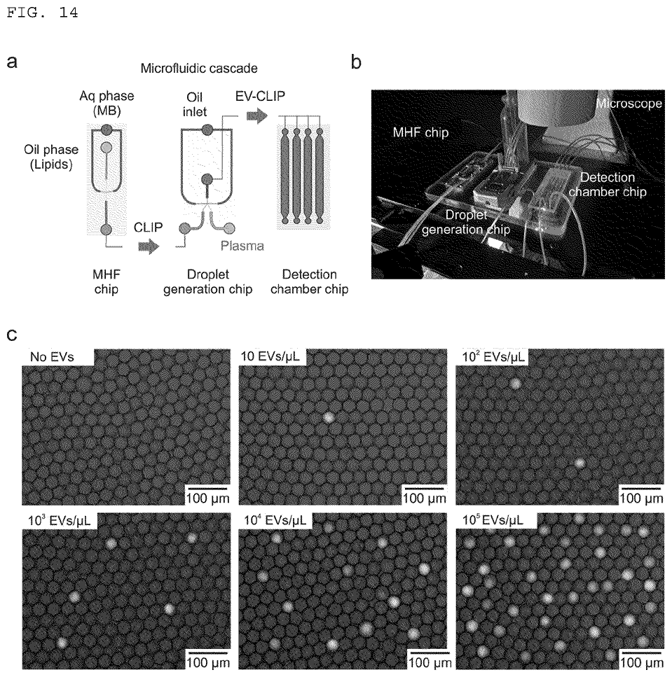

In order to visualize EV-CLIP fusion events, EVs were labeled with green fluorescent dye (3,3′-dioctadecyloxacarbocyanine perchlorate (DiO) dye (ThermoFisher Scientific, V22886)) and CLIPs were labeled with red fluorescent dye (Rhodamine-DHPE (Lissamine rhodamine B 1,2 dihexadecanoyl-sn-glycero-3-phosphoethanolamine, triethylammonium salt)), and confocal laser scanning microscope (CLSM) imaging was performed. CLSM imaging showed the colocalization of dyes within the fused vesicles, consistent with the TEM images and DLS experiments (FIGS. 7d and 7c). In addition, a Forster resonance energy transfer (FRET)-based lipid mixing assay (Stryer, L.; Haugland, R. P., Proc Natl Acad Sci US A 1967, 58 (2), 719-26) was utilized to evaluate the fusion efficiency, CLIPs were dual-labeled with nitro-2,1,3-benzoxadiazole-4-yl (NBD) as a donor and Lissamine rhodamine B (Rho) as an acceptor, and energy transfer occurred from the excited NBD donor to the Rho acceptor when they were in proximity (Francois-Martin, C.; Pincet, F., Scientific Reports 2017, 7 (1), 43860) (FIGS. 7b and 7c). Initially, the effect of dual-labeled lipids on the zeta potential of CLIPs was examined, indicating no significant effect on the surface charge (FIG. 11).

Using the FRET-based assay, an increase in the fusion percentage corresponding to the percentage of DOTAP was observed (FIG. 7c). However, CLIPs containing 100% DOTAP exhibited instability and formed aggregates over time despite having the highest fusion efficiency (FIG. 4). When the fusion method in the droplet reactor was compared with the bulk scale, potential aggregates were revealed (FIG. 12). Consequently, 75% DOTAP was selected as the formulation for subsequent experiments. Also, the ratio of CLIPs to EVs, capable of confirming a maximum fusion percentage of 10:1 (74.96±2.63%), which was further confirmed by CLSM imaging, was adjusted (FIG. 7c).

### Example 4: Digital Detection of Tumor-Derived EVs miRNAs

After successfully demonstrating efficient fusion between CLIPs and EVs, fluorescence response was directly investigated during the fusion process between molecular beacon (MB)-encapsulated CLIP and tumor-derived EV (tEV) miR-21 using a confocal laser scanning microscope (CLSM) to evaluate the effect of the charged-fusion strategy for detecting EV miRNA in natural environments. In this example, the MB utilized a hairpin structure comprising single-stranded oligonucleotide probes inducing fluorescence quenching characterized by a cyanine 3 fluorophore at the 5′ end, a tetrachlorofluorescein quencher at the 3′ end, and complementary bases at both ends that bring the fluorophore and the quencher into close proximity (Zhang, P., et al., Angew Chem Int Ed Engl 2001, 40 (2), 402-405)(Table 2).

TABLE 2
Molecular beacon sequences used for miR-21, EGFR L858R, and
T790M mutations

| Molecular | | SEQ |
|---|---|---|
| beacon | | ID |
| (MB) | Sequence | NO: |
| EFGR (p.T790M | AGCTGC/iCy5/ATGATGAGCTGCACGGTGGCAGCTCATCA | 1 |
| mutation) | T/BHQ2 | |
| EFGR (p.L858R | TTGGCC/iCy3/CGCCCAAAATCTGTGATTAGATTTGGGCG/ | 2 |
| mutation) | BHQ2 | |
| miR-21 | TCAACA/iCy3/TCAGTCTGATAAGCTAGTATTATCAGACTG | 3 |
| | A/BHQ2 | |

Respective underlined parts in Table 2 are the fluorophore and the quencher. The sequence can be referred to Hu, J., et al., Biomaterials, 2018. 183: p. 20-29.

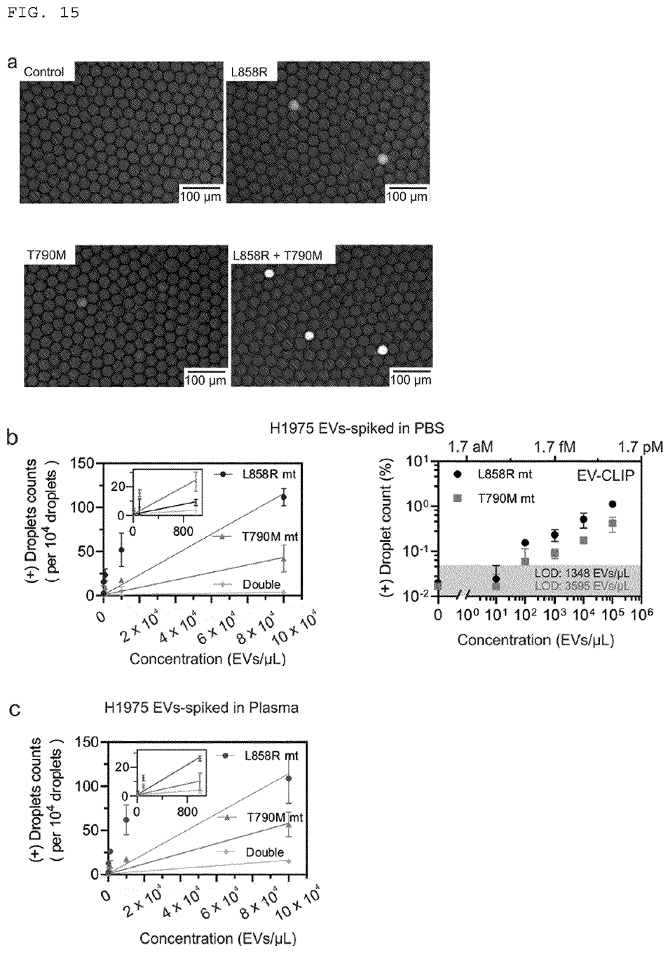

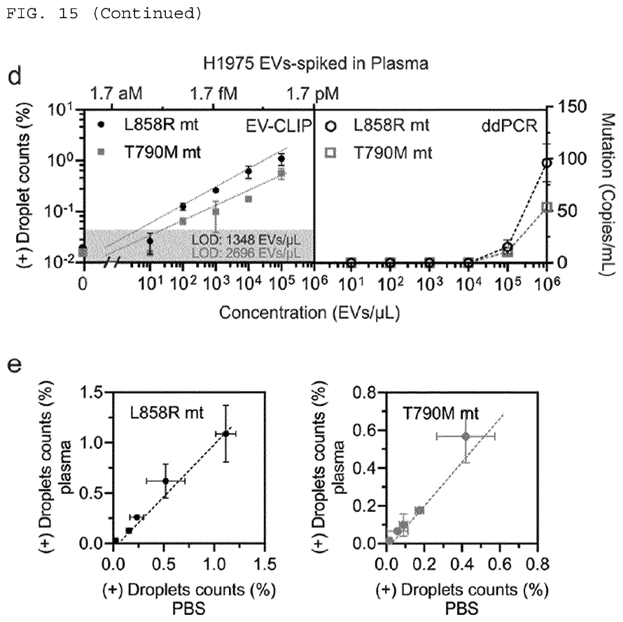

Initially, tumor-derived extracellular vesicles (tEVs) were detected using a molecular probe (MB) specifically designed to target microRNA-21 (or miR-21), which is known to be upregulated in various tumor types, such as breast cancer, colon cancer, lung cancer, pancreatic cancer, prostate cancer, and stomach cancer (Krizhevsky, A. M.; Gabriel, G., J Cell Mol Med 2009, 13 (1), 39-53). During the generation of CLIPs using the microfluidic hydrodynamic focusing method mentioned above, the miR-21-specific MB (miR-21 MB) was loaded by incorporating 1 μM of miR-21 MB into the aqueous phase, and this insertion did not significantly affect the size or zeta potential of the liposomes (FIG. 13b). As a proof of concept, EVs were fused with CLIPs containing miR-21 MB in a droplet reactor and detection of H1975 lung cancer cell line-derived EVs was initiated in phosphate-buffered saline (PBS). The concentration of CLIPs was kept constant throughout the experiment, and varying the concentration of H1975 EVs from 10 to 10⁵ EVs/μl proportionally increased the number of droplets from which fluorescence signals were detected as analyzed through a series of image processing steps (FIGS. 14a-14c).

In order to evaluate system performance, the ability to detect EGFR L858R and T790M mutations (Zhao, B. X., et al., Mol Med Rep 2015, 11 (4), 2767-74) in the H1975 lung cancer cell line was determined. To achieve this, EGFR L858R and T790M mutation-specific MB sequences (Hu, J., et al., Biomaterials 2018, 183, 20-29) were introduced by incorporating 1 μM of each beacon into the aqueous phase using microfluidic hydrodynamic focusing through the same method used to load miR-21 MB. In particular, incorporation of the beacon did not significantly affect the size or zeta potential of CLIPs (FIG. 13).

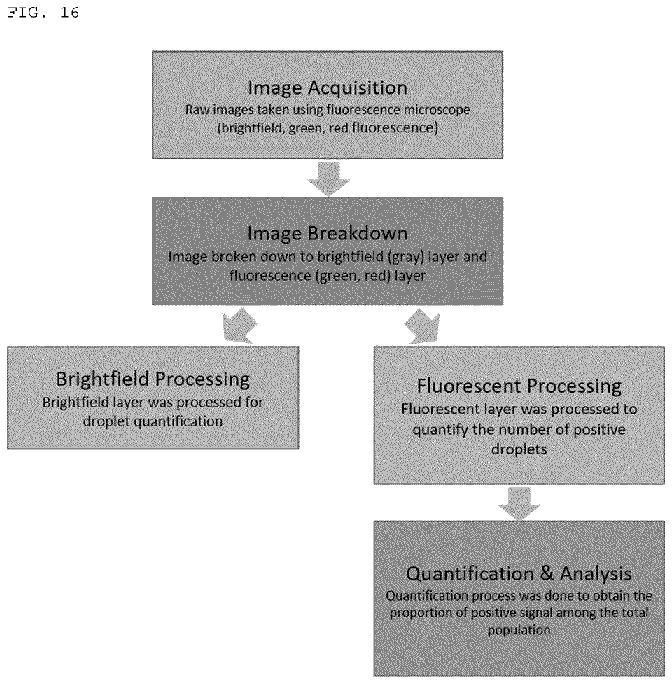

Next, under the same experimental conditions as the previous setup for the droplet reactor generated by the u Encapsulator, with the only difference being the inclusion of specific beacons targeting EGFR L858R and T790M mutations in CLIPs, these mutations were successfully detected. Fluorescence signals were observed only in the presence of target EVs (FIG. 15). Prior to detecting EGFR mutation in blood plasma samples from cancer patients, detection of EGFR L858R and T790M mutations was demonstrated by introducing various concentrations of H1975-derived EVs into healthy human blood plasma (FIGS. 15b-15d). The concentration range was 10 to 10⁵ EVs/μl, and the detection results for both mutations were consistent with those obtained in PBS (FIGS. 15c and 15f), confirming the robustness of the CLIP and EV fusion system. Remarkably, this system is capable of detecting mutation at a level as low as a single EV per 100 nanoliters, which represents at least about 100 times greater sensitivity than conventional methods (FIG. 15d).

### Example 5: Clinical Applicability of EV-CLIP (Charged-Liposome EV Detection) for Patient Sample Analysis

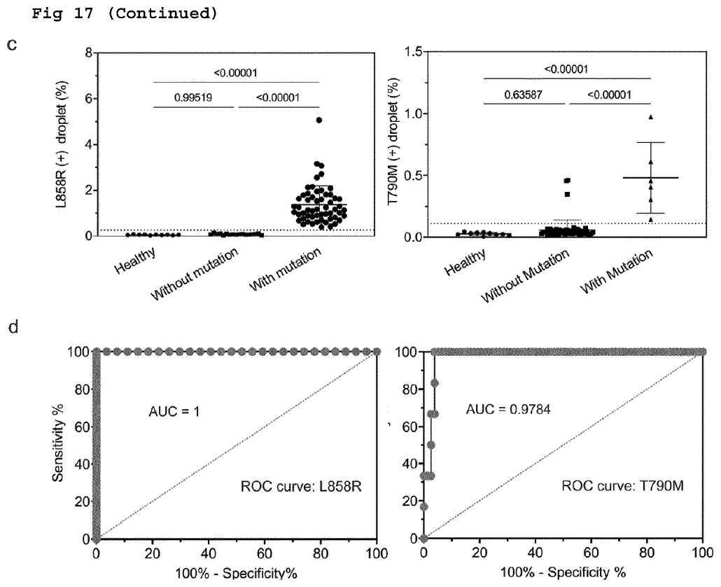

Ultimately, the inventors aimed to put the EV-CLIP system to practical use by testing for EGFR L858R and T790M mutations in blood plasma clinical samples collected from lung cancer patients (FIG. 17a). Plasma samples from 10 healthy donors and 73 lung cancer patients were included (Table 3).

TABLE 3
Patient Information

| | | | Patients | Patients |
|---|---|---|---|---|
| | | Normal | without EGFR | with EGFR |
| | | persons | mutation | mutation |
| Feature | | (n = 10) | (n = 17) | (n = 56) |
| Gender | Female | 5 | 9 | 32 |
| | Male | 5 | 8 | 24 |
| Age | >60 | 4 | 16 | 8 |
| | ≤60 | 6 | 1 | 48 |
| Histological | Adenocarcinoma | 0 | 17 | 53 |
| type | Non- | 0 | 0 | 3 |
| | adenocarcinoma | | | |
| Smoking | Non-smoker | 7 | 0 | 18 |
| status | Smoker | 3 | 0 | 11 |
| | Unknown | 0 | 17 | 27 |
| Mutation | L858R | | 0 | 50 |
| | L858R + T790M | | 0 | 6 |
| Disease | I | 0 | 6 | 18 |
| stage | II | 0 | 3 | 6 |
| | III | 0 | 3 | 7 |
| | IV | 0 | 5 | 25 |

Table 3 shows the basic information of 10 healthy donors and 73 patients used in Examples, including gender, age, histological type, smoking status, mutation, and disease stage: 10 healthy donors, 17 cancer patients without mutation (6 stage I, 3 stage II, 3 stage III, and 5 stage IV), and 56 cancer patients with mutation (18 stage I, 6 stage II, 7 stage III, and 25 stage IV). The samples were provided by Inha University Hospital, Chonnam National University Hwasun Hospital, and Pusan National University Hospital after receiving approval from the Institutional Review Board.

Among lung cancer patients, 17 patients had no mutation and 56 patients had mutations. The detection process was performed without any sample preprocessing. Interestingly, the signal obtained from blood plasma of cancer patients had both L858R and T790M mutations was much higher than the threshold obtained from the ROI curve (FIG. 17d). These results suggest a strong correlation between the presence of these mutations and elevated signal levels in the EV-CLIP system. Regardless of cancer stage, it was observed that only cancer patient samples with positive mutation showed significantly elevated signals. This EV-CLIP method is capable of detecting mutation at a high level in a short time without preprocessing even in early-stage cancer, making it easy to distinguish between cancer patients and healthy donors, as well as to diagnose drug resistance in cancer patients at an early stage.

### Example 6: Materials and Methods

### Example 6-1: Reagents and Equipment

0.2 μm Syringe Filter (Minisart® NML Syringe Filters, S6534), 1× Phosphate-Buffered saline (pH 7.4, Gibco), antibiotic/antimycotic (Gibco, 15240062), EV-depleted FBS (Systems Biosciences Inc), ExoDisc-D20 (LabSpinner), miRNA (Oligo, Bioneer), molecular beacon (Oligo, Bioneer).

0.2 μm syringe filter (Minisart® CA Syringe Filters, 16534K), Encapsulator 2 reagent droplet chip (Dolomite Microfluidics 3200529), 100K Amicon centrifugal filter (Merck Millipore, UFC810096), 150 μm aqueous 5-input chip 3D (Dolomite Microfluidic 3200834), 30 μm chamber chip (Microfluidic ChipShop, 10001447), 96-well plate (Corning, 3364), confocal laser scanning microscope (FV3000, Olympus), NanoSight instrument (Nanosight NS300, Malvern Instruments).

### Example 6-2: Cell Culture

HeLa cells (ATCC) and SH-SY5Y cells (ATCC) were cultured at 37° C. in the presence of 5% CO₂ under static conditions in Dulbecco's Modified Eagle Medium (DMEM) (Gibco, 11965092) supplemented with 10% (v/v) FBS (Gibco) and 1% antibiotic/antimycotic (100 U/ml penicillin and 100 mg/ml streptomycin, Gibco, 15240062).

### Example 6-3: Isolation of Extracellular Vesicle from Cell Culture

Extracellular vesicles were isolated from cell culture medium using a commercialized ExoDisc platform (Woo, H. K., et al., Acs Nano, 2017. 11 (2): p. 1360-1370). 70-80% confluent 10 cm culture dishes of cells were washed twice with 1× phosphate-buffered saline (pH 7.4, Gibco) and then cultured for 48 hours in respective medium supplemented with 5% EV-depleted FBS (Systems Biosciences Inc.) and 1% antibiotic/antimycotic, after which the cell culture supernatant was collected, centrifuged at 300 g for 10 minutes and then at 2,000 g for another 10 minutes to remove cell debris, and passed through a 0.2 μm syringe filter (Minisart® NML Syringe Filters, S6534). Thereafter, the clarified supernatant was concentrated using an ExoDisc platform, a centrifugal disc equipped with anodized aluminum oxide filters with a pore diameter of 0.02 μm (ExoDisc-D20, LabSpinner). The supernatant was centrifuged at 3,000 rpm (approximately 500 g) and passed through the filter, and 100 μl of concentrated EVs from the collection chamber was resuspended in 1×PBS with a dilution factor of 2. Thereafter, the isolated EVs were analyzed using a NanoSight instrument and a Malvern Zetasizer to determine the concentration, size, and zeta potential distribution, and the EVs were aliquoted and stored at −80° C. until they were used for further experiments.

### Example 6-4: Exogenous miRNA Loading into Extracellular Vesicles (EVs) miRNAs (Oligo, Bineer) were loaded into SH-SY5Y cell-derived EVs using the

ExoDisc platform and the protocol (Lee, C., et al., LabChip, 2024, 24: p. 2069-2079). Following the EV separation, a hypotonic solution (0.1 M PBS) containing the loading material was added to the sample chamber of the ExoDisc and rotated for 5 min at 3000 rpm. As the loading materials, we used miRNAs (1 μM, 1 mL). Following the hypotonic exposure, the EVs were washed twice with 1 mL of PBS (1.0 M) to restore them to the isotonic solution and remove any unloaded materials. Optical signals (Absorbance at 260 nm) were measured using a microplate reader (Epoch 2, BioTek) for miRNA concentration, and the loading efficiency was quantified using the following equations:

$$miRNA ( ng / uL ) = {A}_{260} \times dilution factor \times 33 \\ miRNA loading efficiency ( \% ) = \frac{{C}^{inlet} - {C}^{r e s i d u a l}}{{C}^{inlet}} \times 100$$

### Example 6-5: Liposome Synthesis

Liposomes were synthesized by microfluidic hydrodynamic focusing method using a commercialized platform (Dolomite Microfluidics). Lipid mixtures for liposomes were prepared by dissolving various ratios of 18:1 TAP or DOTAP (1,2-diolcoyl-3-trimethylammonium propane, Avanti 890890P) and 18:1 (49-Cis) PC or DOPC (1,2-diolcoyl-sn-glycero-3-phosphocholine, Avanti 850375P)-75% DOTAP—in 1 ml of pure ethanol (Duksan Pure Chemicals, UN1170) filtered using a 0.2 μm syringe filter (Minisart® CA Syringe Filters, 16534K) to reach a final concentration of 5 mg/ml. CLIP synthesis was performed using a 150 μm hydrophilic 5-input chip 3D (Dolomite Microfluidic 3200834) by adjusting the flow rate of an aqueous phase (1X phosphate-buffered saline, pH 7.4, Gibco) to be 50 μl/min and the flow rate of an oil phase (lipid mixture) to be 5 μl/min. The suspension was collected in a 1.5 ml microcentrifuge tube for 15 minutes, purified using a 100K Amicon centrifugal filter (Merck Millipore, UFC810096) with twice PBS washing, and then analyzed using a NanoSight instrument (Nanosight NS300, Malvern Instruments) and a Malvern Zetasizer (Nano ZS) to determine the concentration, size, and zeta potential distribution. Thereafter, CLIPs were aliquoted and stored at 4° C. for further use.

### Example 6-6: Loading of Molecular Beacon into the Liposomes

Molecular beacon design (Table 4) consists of oligonucleotide probes labeled at the 5′ end with a Cy3 fluorophore and at the 3′ end with a BHQ2 quencher, allowing for fluorescence-based detection upon hybridization to target sequences. Each beacon is specific to a particular miRNA (miR-24-1, miR-331-5p, miR-126-5p, miR-504-3p, miR-192-5p, and miR-155-5p shown in Table 5 & 6), with sequences tailored for high specificity and optimal hybridization properties. Table 4 provides key design parameters, including melting temperature (Tm) values ranging from 66.4° C. to 78° C., GC content between 47% and 55%, and Gibbs free energy (ΔG) values at 37° C. from −4.2 to −7.2 Kcal/mol, indicating stable probe-target duplex formation. Alignment scores, which reflect sequence complementarity and specificity, range from 10⁵ to 136, supporting the suitability of these molecular beacons for sensitive and specific microRNA detection.

The miRNA-targeting molecular beacons were loaded into the liposomes by incorporating a certain amount of molecular beacons with a total concentration of 1 μM in 1×PBS. The liposomes were then synthesized using the microfluidic hydrodynamic focusing (MHF) method with a final lipid concentration of 5 mg/ml. The miRNA targeting beacon was loaded into a batch of liposomes. The flow rates of the pumps were maintained at 50 μl/min and 5 μl/min for aqueous and oil phases, respectively. Thereafter, the molecular beacon-loaded liposomes were purified by washing twice with PBS using a 100K Amicon centrifugal filter, and then analyzed using a Nanosight instrument and a Malvern Zetasizer to determine the concentration, size, and zeta potential distribution. The liposomes were then stored at 4° C. for further use.

TABLE 4
Molecular beacons

| | | Tm | GC | ΔG | |
|---|---|---|---|---|---|
| Molecular | | value | content | (at 37° C.) | Alignment |
| beacon | Sequence (5′ → 3′) | (° C.) | (%) | (Kcal/mol) | score |
| miR-24-1 | Cy3/AACGACTGTTCCTGCTGAACTGAG | 70 | 50 | −5.4 | 121 |
| MB | CCATCGTT/BHQ2 (SEQ ID NO: 4) | | | | |
| miR-331-5p | Cy3/TTATTGGATCCCTGGGACCATACC | 66.4 | 47 | −5.1 | 136 |
| MB | TAGCCTAA/BHQ2 (SEQ ID NO: 5) | | | | |
| miR-126-5p | Cy3/CCGAGCCGCGTACCAAAAGTAAT | 76 | 55 | −5.2 | 123 |
| MB | AAGCTCGG/BHQ2 (SEQ ID NO: 6) | | | | |
| miR-504-3p | Cy3/TGATGCAAACCCTGCCCTGCACTG | 78 | 55 | −4.2 | 112 |
| MB | CATCA/BHQ2 (SEQ ID NO: 7) | | | | |
| miR-192-5p | Cy3/TCGGAGGCTGTCAATTCATAGGTC | 75 | 52 | −7.2 | 105 |
| MB | AGTCCGA/BHQ2 (SEQ ID NO: 8) | | | | |
| miR-155-5p | Cy3/ACGCGAACCCCTATCACGATTAGC | 77 | 50 | −5.3 | 120 |
| MB | ATTAACGCGT/BHQ2 (SEQ ID NO: 9) | | | | |

### Example 6-7: Microfluidic Aided EV-CLIP Fusion

Microfluidic Aided fusion was performed using a commercialized platform (Dolomite Microfluidics). The glass chip (u Encapsulator 2 Reagent Droplet Chip, Dolomite Microfluidics 3200529) consists of four channels (two aqueous phase channels, one oil phase channel, and one outlet channel). Liposomes and extracellular vesicles at the same concentration in PBS were loaded into the aqueous channels of the microfluidic chip, and droplets with a diameter of 30 μm were generated in the Fluo-oil 135 (Emulseo) oil phase. The numbers of extracellular vesicles and liposomes can be controlled by varying the initial sample input concentration in the range of 10 to 10⁵ particles/μl, and the pressure of the pumps were maintained at 2,500 mBar and 1,100 mBar for oil and aqueous phases, respectively. The droplets were collected in microcentrifuge tubes for 20 minutes and stored at 4° C. until they were used for further experiments.

### Example 6-8: Detection of Extracellular Vesicle Through Fusion with MB-CLIP

Detection of extracellular vesicles was performed using the microfluidic aided fusion method described above. A fixed concentration of extracellular vesicles (10⁵ particles/μl) and a fixed concentration of molecular beacon containing CLIPs both in 1×PBS, were loaded into the aqueous channels of the microfluidic chip, where droplets of 30 μm in diameter were generated in the Fluo-oil 135 oil phase. The pressures of the pumps were maintained at 2,500 mBar and 1,100 mBar in oil and aqueous phases, respectively. The droplet solution was collected in 1.5 ml brown microcentrifuge tubes for 20 minutes and stored at 4° C. until it is used for further experiments.

### Example 6-9: Droplet Imaging

For imaging, 6 μl of the droplet solution was loaded into a 30 μm chamber chip (Microfluidic ChipShop, 10001447) and the droplets were observed in a confocal laser scanning microscope (Olympus FV3000) using a ×10 Plan-Apochromat and a 0.45 NA objective lens equipped with a diode laser unit 561 nm (red/Cy3) and GaAsP photomultiplier. All images for droplet analysis were processed using in-house made image SW.

### Example 7: Cell and Cell-Derived EV Characteristics

HeLa cell is an immortalized human cancer cell line. In neurodegenerative studies, HeLa cell-derived EVs serve as a non-neuronal reference (or negative control) to contrast with EVs from disease models because HeLa-derived EVs don't naturally contain AD/PD/HD-associated genes or proteins (e.g. amyloid-β, tau, or mutant huntingtin), making them useful for isolating disease-specific EV effects.

SH-SY5Y cell, derived from human neuroblastoma, is a widely used in vitro model for studying neurodegenerative diseases like Alzheimer's (AD), Parkinson's (PD), and Huntington's disease (HD). These cells can differentiate into neuron-like phenotypes, express disease-relevant markers, and serve as platforms for EV-based research. In this invention, SH-SY5Y cell had not been differentiated during culture for being used as normal cells and EVs. Undifferentiated SH-SY5Y cell, and EVs loaded with exogenous miRNAs, are increasingly employed to validate disease mechanisms and therapeutic strategies (Menjivar, N. G., et al., Biol Proced Online, 2024, 26 (14)).

### Example 8: MiRNA and miRNA-Loaded EV Characterization

### Example 8-1: MiRNAs

In the present disclosure, 6 different miRNAs were selected to make neurodegenerative disease models (AD, PD, and HD).

miR-24-1 is experimentally linked to Parkinson's disease (PD), showing specificity for PD samples, though its mechanistic role in neurodegeneration remains underexplored in published studies. miR-331-5p is associated with Alzheimer's disease (AD), where it impairs autophagy by targeting SQSTM1 and OPTN, leading to toxic protein accumulation (e.g., Aβ in AD). miR-126-5p is a multi-disease biomarker (AD/PD/HD) with neuroprotective roles in angiogenesis and endothelial survival, though its broad expression reduces diagnostic specificity. miR-504-3p is tied to AD and Huntington's disease (HD), where it modulates tau hyperphosphorylation via p39 inhibition, a regulator of CDK5 activity. miR-192-5p is linked experimentally to AD, potentially influencing APP expression, reducing Ab accumulation. miR-155-5p drives neuroinflammation in AD/PD by promoting neuroinflammation via microglial activation and enhancing a-synuclein-induced inflammation.

TABLE 5
Neurodegenerative disease-specific miRNAs

| | Target | | |
|---|---|---|---|
| miRNA | Disease | Target mRNA/Protein | Functional Role |
| miR-24-1 | PD | Not explicitly identified | Upregulated in PD serum; potential biomarker |
| | | | for differential diagnosis vs. MSA |
| miR-331-5p | AD | SQSTM1, OPTN | Regulates autophagy; biphasic expression |
| | | | (downregulated early, upregulated late in AD) |
| miR-126-5p | AD, PD, | AD: PI3K/ERK pathways | AD: Modulates Aβ toxicity via GF signaling |
| | HD | PD: SP1 | PD: Reduces apoptosis/inflammation |
| | | HD: Not specified | HD: Downregulated in HD cell models |
| miR-504-3p | AD, HD | AD: CDK5R1 (p39) | AD: Reduces tau hyperphosphorylation |
| | | HD: ATXN1 | HD: Upregulated in HD; |
| | | | targets ATXN1 (spinocerebellar ataxia gene) |
| miR-192-5p | AD | AD: APP (*Amyloid | AD: Downregulates APP expression, reducing |
| | | precursor protein) | Aβ accumulation |
| miR-155-5p | AD, PD | AD/PD: SOCS1 | AD: Promotes neuroinflammation via |
| | | | microglial activation |
| | | | PD: Enhances α-synuclein-induced |
| | | | inflammation |

6 miRNA oligonucleotides were synthesized and purified by HPLC purification (Bioneer). All oligonucleotides were quantified by optical density (OD260) measurement and water content analysis.

TABLE 6
miRNA sequences

| miRNA | Sequence (5′ → 3′) |
|---|---|
| miR-24-1 | CUCCGGUGCCUACUGAGCUGAUAUCAGUUCUCAUUUUACACACUGGCUC |
| | AGUUCAGCAGGAACAGGAG (68 mer) (SEQ ID NO: 10) |
| miR-331-5p | CUAGGUAUGGUCCCAGGGAUCC (22 mer) (SEQ ID NO: 11) |
| miR-126-5p | UUAUUACUUUUGGUACGCG (19 mer) (SEQ ID NO: 12) |
| miR-504-3p | AGUGCAGGGCAGGGUUU (17 mer) (SEQ ID NO: 13) |
| miR-192-5p | CUGACCUAUGAAUUGACAGCC (21 mer) (SEQ ID NO: 14) |
| miR-155-5p | UUAAUGCUAAUCGUGAUAGGGGUU (24 mer) (SEQ ID NO: 15) |

### Example 8-2: MiRNA-Loaded EV Characteristics

SH-SY5Y cell-derived EVs were loaded by each miRNA exogenously based on the published reference (Lee, C., et al., LabChip, 2024, 24: p. 2069-2079) and characterized for EV's size distribution by NTA (Nanosight NS300) before and after miRNA loading, and miRNA loading efficiency by the equation (See Example 6-4).

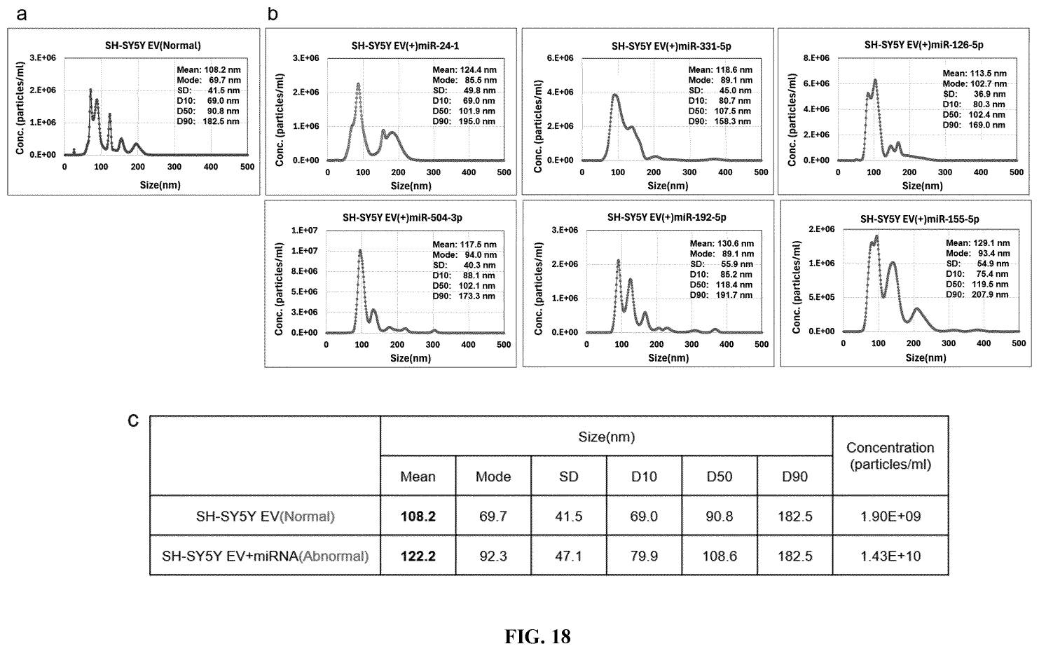

FIG. 18 shows the EV size distributions for naive SH-SY5Y cell-derived EVs (FIG. 18a) and miRNA-loaded EVs for each miRNA (FIG. 18b). Measurements were performed in triplicate. Significant differences in size distributions were observed between naive EVs and miRNA-loaded EVs. The mean EV size increased by 12.9% (from 108.2 nm to 122.2 nm), while the mode size rose by 23.4% (from 69.7 nm to 92.3 nm), with comparable standard deviations across groups (FIG. 18c).

To quantify miRNA loading efficiency, input (or initial) and residual concentration of miRNAs were measured before and after miRNA loading into EVs by using a microplate reader (Epoch 2, BioTek). The miRNA loading efficiency was calculated by 33% of the initial miRNA input.

These findings demonstrate the successful loading of miRNAs into SH-SY5Y cell-derived EVs.

### Example 9: Digital Detection of Neurodegenerative Disease-Specific miRNAs

### Example 9-1: Imaging Droplets

For imaging, 6 ml of the droplet solution was loaded into a 30-mm chamber chip (Microfluidic ChipShop) and the confocal microscope (Olympus FV3000) was set up by an optimized condition (10× lens, 561 nm-laser, HV 850, Gain 1.25×, Laser power 20%, resolution 512×512).

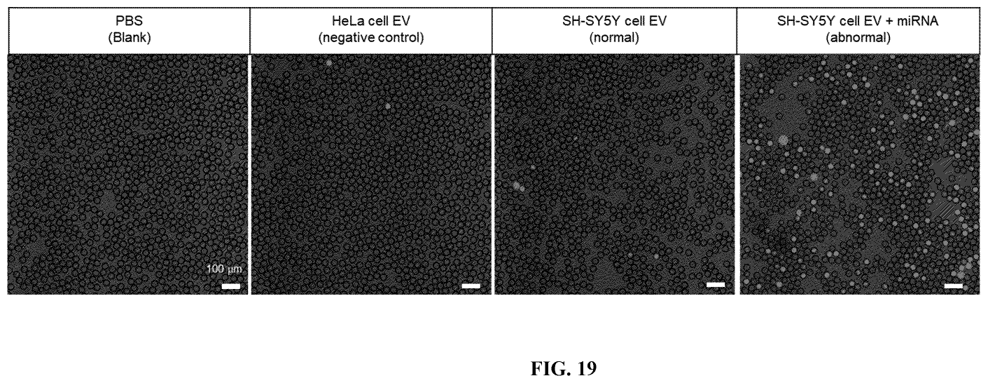

FIG. 19 shows a representative image combined by bright field image for droplets and fluorescence image for 4 different kinds of test samples: PBS (as a blank), HeLa cell EV (negative control), SH-SY5Y cell EV (normal), and SH-SY5Y cell EV±miRNA (abnormal). The red-colored droplets have specific miRNAs, indicating positive signal, whereas non-red droplets don't have those miRNAs, indicating negative signal. The number of each test sample were 20 replicates.

### Example 9-2: Clustering Droplet Images by AI Algorithms

One of the best methods to differentiate negative and positive signals of the droplet images is clustering AI algorithms. Key algorithms were MINMAX for normalization, CLAHE (Contrast Limited Adaptive Histogram Equalization), embedding for clustering, and dimension reducing (from 2048 to 2 dimensions).

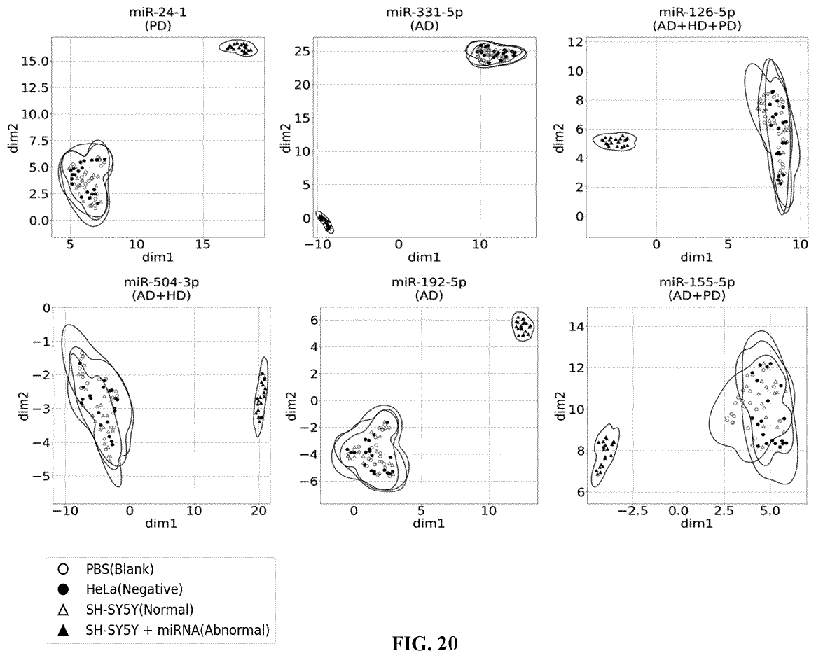

FIG. 20 shows clustering analyses performed using dimensionality reduction on experimental data from four test sample groups-PBS (blank), HeLa (negative control), SH-SY5Y (normal), and SH-SY5Y±miRNA (abnormal)—for six different miRNAs relevant to neurodegenerative diseases. In each case, the abnormal (SH-SY5Y±miRNA) samples formed distinct and well-separated clusters from the normal and control groups, regardless of the specific miRNA tested. This clear separation demonstrates the assay's ability to discriminate between normal and miRNA-contained cellular states, supporting the utility of the method for detecting disease-associated molecular changes in a robust and reproducible manner.

### Example 9-3: Quantifying Amount of miRNA Contained in EVs

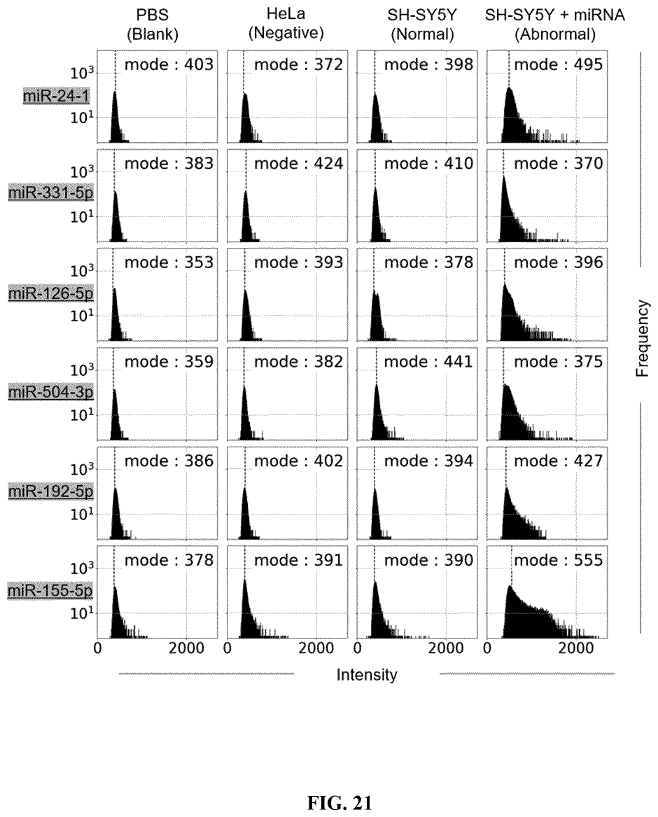

The first step involved quantifying EV miRNAs in test samples (N=20 per group) by analyzing droplet fluorescence intensity and generating histograms. As illustrated in FIG. 21, the mean fluorescence intensity per droplet was calculated across all images (N=15-20 per test), and intensity distributions were plotted for each sample type and miRNA. Histograms revealed that abnormal samples (SH-SY5Y±miRNA) exhibited the highest skewness in their Poisson distributions compared to normal and control groups, a trend consistent across all tested miRNAs. This deviation suggests a distinct miRNA expression profile in abnormal conditions.

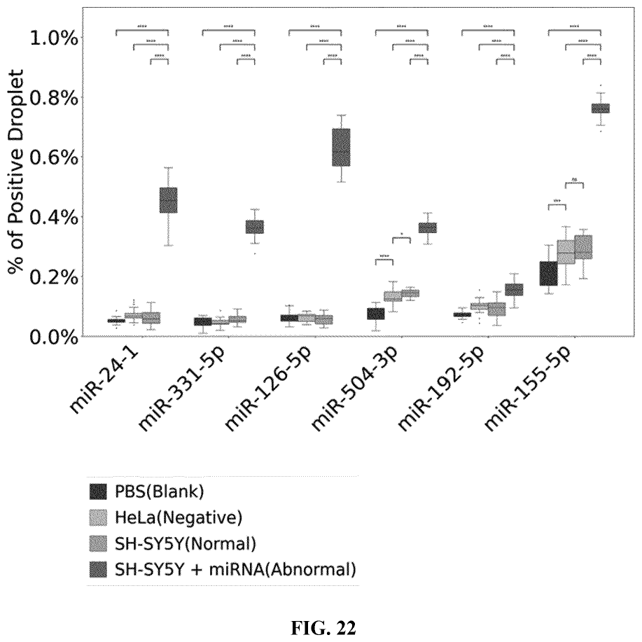

FIG. 22 demonstrates the second quantification step, where % positive droplets served as a proxy for miRNA concentration. While this metric is relative, absolute concentrations can be extrapolated using a standard curve correlating % positive droplets with EV miRNA levels measured by conventional methods (e.g., RT-PCR). Abnormal samples showed statistically significant differences in % positive droplets compared to blank and controls (p≤0.0001), underscoring the method's sensitivity in differentiating miRNA profiles.

### Example 9-4: Correlation of Neurodegenerative Disease Based on EV miRNA Expressions

The present study quantified extracellular vesicle (EV) miRNAs via the EV-CLIP digital detection method, identifying neurodegenerative disease-specific miRNAs as detailed in Tables 5 and 6.

Table 7 presents three disease-discrimination equations derived from multivariate linear regression analysis. These models utilize miRNA expression data to distinguish Alzheimer's disease (AD), Parkinson's disease (PD), and Huntington's disease (HD). Performance validation using 5-fold cross-validation yielded R² scores (coefficient of determination) for each model.

These findings demonstrate the potential of the EV-CLIP method for quantifying neurodegenerative disease biomarkers in diagnostic or monitoring applications. However, further validation with larger sample sizes and clinical correlation studies is required to confirm the models' robustness.

TABLE 7
Disease discrimination equations

| Disease | Equation |
|---|---|
| AD | AD = −0.0012 · miR-24-1 + 0.0055 · miR-331-5p + 0.0033 · miR-126-5p − 0.0039 · miR-504- |
| | 3p + 0.0185 · miR-192-5p − 0.0011 · miR-155-5p (R² = 0.72 ± 0.08) |
| PD | PD = 0.0037 · miR-24-1 − 0.0021 · miR-331-5p + 0.0019 · miR-126-5p − 0.0023 · miR-504-3p − 0.0092 · miR− |
| | 192-5p + 0.0024 · miR-155-5p (R² = 0.65 ± 0.11) |
| HD | HD = −0.0014 · miR-24-1 + 0.0008 · miR-331-5p + 0.0015 · miR-126-5p + 0.0031 · miR-504- |
| | 3p − 0.0043 · miR-192-5p + 0.0006 · miR-155-5p (R² = 0.58 ± 0.13) |

### INDUSTRIAL APPLICABILITY

According to the present invention, charged-liposome EV detection (EV-CLIP) can be very usefully applied in clinical settings by providing accurate quantification of rare EV subpopulations and opening new avenues for exploring fundamental questions in cancer biology. In addition to direct applications in diagnostics, such a fusion system has tremendous potential for a variety of implementations, including drug delivery carrier development, EV marker profiling, and many other possibilities.

Having described certain parts of the present invention in detail above, it will be obvious to those skilled in the art that these specific descriptions are only preferred embodiments, and the scope of the present invention is not limited thereby. Accordingly, the substantial scope of the present invention will be defined by the appended claims and equivalents thereto.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A liposome for detecting a pathological cell-derived extracellular vesicle (EV) from a subject with a neurodegenerative disease comprising a cationic lipid and a neutral lipid, in which a pathological cell-specific molecular beacon is encapsulated within the liposome.

2. The liposome according to claim 1, wherein the cationic lipid is selected from the group consisting of 1,2-diolcoyl-3-trimethylammonium propane (DOTAP), 1,2-dioleyloxy-3-dimethylamino-propane (DODMA), 1,2-di-O-octadecenyl-3-trimethylammonium propane (DOTMA), 3β-[N-(N′,N′-dimethylaminoethane)-carbamoyl]cholesterol (DC-Chol), dimethyldioctadecylammonium bromide (DODAB), 1,2-diolcoyl-3-dimethylammonium-propane (DODAP), dioctadecyldimethyl ammonium chloride (DODAC), 1,2-distearyloxy-N,N-dimethyl-3-aminopropane (DSDMA), 1,2-dimyristyloxy-propyl-3-dimethyl-hydroxyethyl ammonium bromide (DMRIE), 1,2-dimyristoyl-sn-glycero-3-ethylphosphocholine (DMEPC), 1,2-dimyristoyl-3-trimethylammonium propane (DMTAP), 1,2-dioleoyl-3-dimethyl-hydroxyethyl ammonium bromide (DORIE), 2,3-dioleyloxy-N-[2-(sperminecarboxamido) ethyl]-N,N-dimethyl-1-propanaminium (DOSPA), 1,2-dilinoleyloxy-N,N-dimethyl-3-aminopropane (DLinDMA), 1,2-dilinolenyloxy-N,N-dimethyl-3-aminopropane (DLenDMA), dioctadecylamidoglycylspermine (DOGS), 3-dimethylamino-2-(cholest-5-en-3-β-oxybutan-4-oxy)-1-(cis,cis-9,12-octadecadienoxy) propane (CLinDMA), 2-[5′-(cholest-5-en-3-β-oxy)-3′-oxapentoxy]-3-dimethyl-1-(cis,cis-9′, 12′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-dilinoleoyloxy-N,N-dimethylpropylamine (DLinDAP), 1,2-N,N′-dilinoleylcarbamyl-3-dimethylaminopropane (DLincarbDAP), 1,2-dilinoleoylcarbamyl-3-dimethylaminopropane (DLinCDAP), 2,2-dilinoleyl-4-dimethylaminomethyl-[1,3]-dioxolane (DLin-KDMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino) butanoate (DLin-MC3-DMA), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), (±)-N-(2-aminoethyl)-N,N-dimethyl-2,3-bis(tetradecyloxy)-1-propanaminium bromide (BAE-DMRIE), N-(4-carboxybenzyl)-N,N-dimethyl-2,3-bis(oleoyloxy) propan-1-aminium (DOBAQ), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido) ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-aminium bromide (DLRIE), N-(2-aminoethyl)-N,N-dimethyl-2,3-bis(tetradecyloxy) propan-1-aminium bromide (DMORIE), di((Z)-non-2-en-1-yl) 8,8′-((((2 (dimethylamino)ethyl) thio) carbonyl) azanediyl) dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), di((Z)-non-2-en-1-yl)-9-((4-(dimethylaminobutanoyl)oxy) heptadecanedioate (L319), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino) propionamide (lipidoid 98N12-5), and 1-[2-[bis(2-hydroxydodecyl)amino]ethyl-[2-[4-[2-[bis(2-hydroxydodecyl)amino]ethyl]piperazin-1-yl]ethyl]amino]dodecan-2-ol (lipidoid C12-200).

3. The liposome according to claim 1, wherein the neutral lipid is selected from the group consisting of 1,2-dioleoyl-sn-glycero-3-phosphocholine (DOPC), 1,2-dimyristoyl-sn-glycero-3-phosphocholine (DMPC), 1,2-dioleoyl-sn-glycero-3-phosphoethanolamine (DOPE), 1,2-dipalmitoyl-sn-glycero-3-phosphocholine (DPPC), 1,2-distearoyl-sn-glycero-3-phosphocholine (DSPC), 1,2-dilinoleoyl-sn-glycero-3-phosphocholine (DLPC), phosphatidylserine (PS), phosphatidylethanolamine (PE), phosphatidylglycerol (PG), phosphoric acid (PA), and phosphatidylcholine (PC).

4. The liposome according to claim 1, wherein the cationic lipid is 1,2-dioleoyl-3-trimethylammonium propane (DOTAP) and the neutral lipid is 1,2-dioleoyl-sn-glycero-3-phosphocholine (DOPC).

5. The liposome according to claim 1, wherein a molar ratio (%) of the cationic lipid in the liposome is 25 to 75%.

6. A composition for detecting a pathological cell-derived extracellular vesicle comprising the liposome according to claim 1.

7. A kit for detecting a pathological cell-derived extracellular vesicle comprising the liposome according to claim 1.

8. A method of detecting a pathological cell-derived extracellular vesicle, comprising:
fusing the liposome according to claim 1 with an extracellular vesicle derived from a biological sample; and
determining that the extracellular vesicle is a pathological cell-derived extracellular vesicle when a fluorescence signal is generated.

9. The method according to claim 8, wherein the fusing is performed within a droplet reactor comprising two aqueous phase channels, one oil phase channel, one junction, and one outlet channel.

10. The method according to claim 9, wherein aqueous droplets are generated at the junction by introducing the liposome and the extracellular vesicle derived from the biological sample respectively into the two aqueous phase channels.

11. A diagnostic composition comprising the liposome according to claim 1.

12. The liposome according to claim 1, wherein the neurodegenerative disease is selected from the group consisting of Huntington's disease, Alzheimer's disease, and Parkinson's disease.

13. A kit comprising the liposome according to claim 1 and instructions for use thereof.

14. The kit according to claim 13, wherein the instructions for use comprise diagnosing a neurodegenerative disease selected from the group consisting of Huntington's disease, Alzheimer's disease, and Parkinson's disease.

15. A method of selecting a subject having neurodegenerative disease for treatment, comprising:
fusing the liposome according to claim 1 with an extracellular vesicle derived from a biological sample; and
selecting the subject for treatment when a fluorescence signal is generated.

16. The method according to claim 15, wherein the fusing is performed within a droplet reactor comprising two aqueous phase channels, one oil phase channel, one junction, and one outlet channel.

17. The method according to claim 15, wherein the neurodegenerative disease is selected from the group consisting of Huntington's disease, Alzheimer's disease, and Parkinson's disease.

18. The liposome according to claim 1, wherein the molecular beacon comprises a nucleic acid and a fluorophore.

19. The liposome according to claim 18, wherein the nucleic acid comprises a sequence encoding one or more of SEQ ID NOs: 4-9.
